SEQUENCE LISTING (18 sequences)

SEQ ID NO: 1 - protein, 84 aa, Artificial Sequence
  Description of Artificial Sequence Synthetic peptide
PRGPPGPPGKPGDDGEAGKPGKSGERGPPGPQGARGFPGTPGLPGVKGHRGYPGLDGAKGEAGAPGVKGESGSPGQNGSPGGPM

SEQ ID NO: 2 - protein, 31 aa, Artificial Sequence
  Description of Artificial Sequence Synthetic peptide
GPRGPPGPPGKPGDDGEAGKPGKSGERGPPG

SEQ ID NO: 3 - protein, 23 aa, Artificial Sequence
  Description of Artificial Sequence Synthetic peptide
ERGPPGPQGARGFPGTPGLPGVK

SEQ ID NO: 4 - protein, 25 aa, Artificial Sequence
  Description of Artificial Sequence Synthetic peptide
GLPGVKGHRGYPGLDGAKGEAGAPG

SEQ ID NO: 5 - protein, 24 aa, Artificial Sequence
  Description of Artificial Sequence Synthetic peptide
GEAGAPGVKGESGSPGQNGSPGPM

SEQ ID NO: 6 - protein, 24 aa, Artificial Sequence
  Description of Artificial Sequence Synthetic peptide
GERGPPGPQGARGFPGTPGLPGVK

SEQ ID NO: 7 - protein, 24 aa, Artificial Sequence
  Description of Artificial Sequence Synthetic peptide
GERGPPGPQGARGFPGTPGLPGVK

SEQ ID NO: 8 - protein, 24 aa, Artificial Sequence
  Description of Artificial Sequence Synthetic peptide
GERGPPGPQGARGFPGTPGLPGVK

SEQ ID NO: 9 - protein, 24 aa, Artificial Sequence
  Description of Artificial Sequence Synthetic peptide
GERGPPGPQGARGFPGTPGLPGVK

SEQ ID NO: 10 - protein, 10 aa, Artificial Sequence
  Description of Artificial Sequence Synthetic peptide
GKSGERGPPG

SEQ ID NO: 11 - protein, 85 aa, Artificial Sequence
  Description of Artificial Sequence Synthetic peptide
GPRGPPGPPGKPGDDGEAGKPGKSGERGPPGPQGARGFPGTPGLPGVKGHRGYPGLDGAKGEAGAPGVKGESGSPGQNGSPGGPM

SEQ ID NO: 12 - protein, 85 aa, Artificial Sequence; identical to SEQ ID NO: 11
  Description of Artificial Sequence Synthetic peptide

SEQ ID NO: 13 - protein, 24 aa, Artificial Sequence
  Description of Artificial Sequence Synthetic peptide
GERGPPGPQGARGFPGTPGLPGVK

SEQ ID NO: 14 - protein, 24 aa, Artificial Sequence
  Description of Artificial Sequence Synthetic peptide
GERGPPGPQGARGFPGTPGLPGVK

SEQ ID NO: 15 - protein, 31 aa, Artificial Sequence; identical to SEQ ID NO: 2
  Description of Artificial Sequence Synthetic peptide

SEQ ID NO: 16 - protein, 25 aa, Artificial Sequence
  Description of Artificial Sequence Synthetic peptide
GLPGVKGHRGYPGLDGAKGEAGAPG

SEQ ID NO: 17 - protein, 24 aa, Artificial Sequence
  Description of Artificial Sequence Synthetic peptide
GEAGAPGVKGESGSPGQNGSPGPM

SEQ ID NO: 18 - protein, 84 aa, Artificial Sequence
  Description of Artificial Sequence Synthetic peptide
GPRGPPGPPGKPGDDGEAGKPGKSGERGPPGPQGARGFPGTPGLPGVKGHRGYPGLDGAKGEAGAPGVKGESGSPGQNGSPGPM

(19) United States
(12) Patent Application Publication
(10) Pub. No.: US 2004/0122209 A1
(43) Pub. Date: Jun. 24, 2004

(54) Products for regulating the degradation of collagen and methods for identifying same

(75) Inventor: A. Robin Poole, South Lancaster (CA)

Correspondence Address: FOLEY AND LARDNER, SUITE 500, 3000 K STREET NW, WASHINGTON, DC, 20007, US

(73) Assignee: Shriners Hospital For Children

(21) Appl. No.: 10/674,065

(22) Filed: Sep. 30, 2003

Related U.S. Application Data
(60) Provisional application No. 60/414,332, filed on Sep. 30, 2002.

(51) Int. Cl. A61K 38/17; C07K 14/47

(52) U.S. Cl. 530/324; 514/12

(57) ABSTRACT

The present invention provides products and methods for regulating the degradation of collagen, including type II collagen. Also encompassed are variants, inhibitors, and mimetics of type II collagen peptide fragments and inhibitors of the proteases producing these peptide fragments that are capable of modifying the degradation of collagen whereby the pathological effects of increased collagen destruction are reduced. In addition, the present invention provides methods for treating disease states wherein the disease state results directly or indirectly from the degradation of one or more collagen species. Furthermore, the present invention encompasses the screening of these peptide fragments for diagnostic purposes.

### CROSS REFERENCE TO RELATED APPLICATIONS

This application is based on U.S. provisional patent application serial No. 60/414,332, filed Sep. 30, 2002. The entire contents of this application, including its specification, claims and drawings, are incorporated herein by reference in their entirety.

### FIELD OF THE INVENTION

This invention relates to products and methods for regulating the degradation of collagen, including type II collagen. More particularly the invention relates to the discovery that unique peptide fragments of type II collagen have an auto regulatory function and can modulate both cell differentiation and the degradation of collagen in vitro and in vivo. Also encompassed within the scope of the invention are variants, inhibitors and mimetics of these peptide fragments and inhibitors of the proteases producing these peptide fragments which are capable of modifying the degradation of collagen so as to reduce the pathological effects of increased collagen destruction. These compounds are useful in the treatment of disease states wherein the disease state results directly or indirectly from the degradation of one or more species of collagen. The invention also encompasses the screening for peptide fragments of the invention for diagnostic purposes.

### BACKGROUND OF THE INVENTION

The physiological turnover of collagen, for example within the extracellular matrix of the articular cartilage, represents a balance between synthesis and degradation. This balance is a feature of normal growth and development and maintenance of cartilage in the adult. Net collagen destruction, however, with ensuing loss of cartilage and joint function, is a feature of many forms of arthritis including, osteoarthritis (OA), adult and juvenile rheumatoid arthritis (RA), post-traumatic OA, and idiopathic OA, psoriatic arthritis, and ankylosing spondylitis. Other diseases also result from the abnormal turnover of collagen including eye diseases and fibrosis; including lung diseases, such as chronic obstructive pulmonary disease, and skin diseases, such as scleroderma. The molecules involved which activate or increase collagen turnover, have not, to date, been well understood.

Collagen Fibrils

Approximately 25 different collagenous polypeptides (α-chains) have been identified. See Kielty, C. M., Hopkinson I, Grant M E. Collagen, the collagen family: structure, assembly and organization in the extracellular matrix. In Royce, P. M., Steinmann, B. (ed): Connective Tissue and its Heritable Disorders. Molecular, Genetic and Medical Aspects, pp. 103-147, New York, Wiley-Liss, 1993. These polypeptides occur in at least 19 different collagen types, designated type I through type XIX. These tropocollagen molecules are best defined structurally: collagen is a molecule comprising three polypeptides (α-chains), which fold to form triple-helical and non-helical domains. This helical structure is determined by the high glycine and imino acid contents in specific repeating triplets of -Gly-X-Y, where X is often proline and Y is often hydroxyproline, which assemble into supramolecular aggregates in the extracellular matrix.

In the case of types I, II, and III collagens, tropocollagen triple helices assemble into fibrils. These fibrillar collagen molecules are visible by electron microscopy. Collagen fibrils consist of parallel quarter staggered alignments of tropocollagen. See Kielty et al. supra.

Type II Collagen

The type II collagen fibril contains 300 nm long type II tropocollagen molecules (each of which contains a triple helix of three identical α-chains), with nonhelical amino and carboxyl terminal telopeptide domains. The association of these collagen molecules is stabilized and strengthened by hydroxy pyridinoline and pyridinoline cross-links. See Mayne, R. What is collagen? In: Koopman W J, (ed): Arthritis and Allied Conditions: A Textbook of Rheumatology, 14^{th} Edition, pp. 187-208, Lippincott, Williams & Wilkins, Philadelphia, 2001.

Type II collagen is the most predominant form of collagen, in articular cartilage comprising of 15-25% of the wet weight of the extracellular matrix and 90-95% of the total collagen content of the cartilage. See Poole, A. R. et al., Clin. Orthop. 391:S26-33 (2001) and Mayne R. supra. Type II collagen endows articular and other hyaline cartilages with their tensile properties. Type II cartilage is also the major collagenous component of vitreous humor. See Kielty et al. supra.

The correct organization of type II collagen within the extracellular matrix of cartilage is essential for the normal function of the matrix. Procollagen bearing the amino (N) and carboxy (C) propeptides is secreted from chondrocytes into the extracellular matrix, where it forms fibrils with the removal of C- and N-propeptides by specific C- and N-proteinases. See Kielty et al. supra and Mayne, R. supra.

In osteoarthritic cartilage there is a loss of the tensile properties, indicating damage to the fibrillar network. See Poole, A. R. Cartilage in Health and Disease. In W. J. Koopman (ed.): Arthritis and Allied Conditions: A Textbook of Rheumatology, 14^{th} Ed., Vol. 1., pp. 226-284, Lippencott, Williams,& Wilkins, Philadelphia, 2001a and Poole A. R. and Howell D. S. Etiopathogenesis of Osteoarthritis. In R. W. Moskowitz et al. (ed): Osteoarthritis: Diagnosis and Medical Surgical Management, 3^{rd} Edition, pp. 29-47, Saunders Company, Philadelphia, 2001b.

In early experimental OA models in dogs, there is a progressive loss of the tensile modulus and a loss of type II collagen content. See Guilak F. et al., J. Orthop. Res. 4:474-84 (1994); and Setton, L. A. et al., J. Orthop. Res. 4:451-63 (1994). This tensile modulus and loss of type II collagen was also noted in human OA. See Akisuki, S. et al., J. Orthop. Res. 4:379-92 (1986); and Hollander, A. P. et al., J. Clin. Invest. 6:2859-69 (1995). Furthermore, it is known that abnormalities in the helical structure of type II collagen, resulting from a mutation in the COL2A1 gene, causes an altered helical structure and can cause premature cartilage degeneration leading to the presentation of familial OA. See Eyre, D. R. et al., J. Rheumatol. Suppl. 27:49-51 (1991); Knowlton, R. G. et al., New Engl. J. Med. 322(8):526-30 (1990); Ritvaniemi, P. et al., Arthritis Rheum. 38(7):999-1004 (1995); and Poole, A.R. (2001a) supra.

Since collagen is mainly responsible for the tensile properties of cartilage, the researchers have suggested that progressive damage to type II collagen results in clinical disease states involving joint destruction and damage to hyaline cartilages and tissues containing type II collagen including osteoarthritis (OA), rheumatoid arthritis (RA), juvenile osteoarthritis juvenile OA), post-traumatic osteoarthritis (post-traumatic OA), idiopathic osteoarthritis (idiopathic OA), psoriatic arthritis, and ankylosing spondylitis. See Kempson, G. E. et al., Biochim. Biophys. Acta. 297:456-72 (1973). Degeneration of type II collagen in the eye may be involved in diseases of the eye. Although type II collagen degradation can occur as part of the natural aging process (Poole, A. R. (2001a) supra), it is thought that beyond a certain critical point such degradation results in the clinical disease states such as joint degeneration in OA mentioned above. See Wu, W. et al., Arthritis Rheumatism, 46:2087-2094 (2002).

In the development of OA in humans, type II collagen is increasingly denatured See Hollander, A. P. et al. (1995) supra. This occurs in association with increased cleavage of type II collagen by collagenases. See Billinghurst, R. C. et al., J. Clin. Invest. 99:1534-45 (1997) and Dahlberg, L. et al. Arthritis Rheum. 43(3):673-82 (2000). This damage no doubt accounts for the loss of tensile properties described above since the collagen fibril determines these properties. See Poole, A. R. (2001 a) supra and Poole A. R. and Howell D. S. (2001b) supra. Denaturation of collagen leads to a loss of triple helical structure and the resultant exposure of α-chain sequences that are ordinarily masked in the triple helical structure. See Dodge, G. R. and Poole, A. R., J. Clin. Invest. 83(2):647-61 (1989) and Hollander, A. P. et al., J. Clin. Invest. 93:1722-32 (1994).

Proteinases Involved in the Degradation of Cartilage

The proteinases known to play a role in the degradation of extracellular matrix are the metalloproteases (MMPs), cysteine proteases and serine proteases. See Poole, A. R. (2001a) supra; and Mort, J. S. and Poole, A. R. Mediators of Inflammation, Tissue Destruction, and Repair. D. Proteases and their Inhibitors. In J. H. Klippel, L. J. Crofford, J. H. Stone, and C. M. Weyand (ed.): Primer on the Rheumatic Diseases, 12^{th} ed., Volume 88, pp 72-81, Arthritis Foundation, Atlanta, Ga., 2001. MMPs are generally considered to play a principal role in the final degradative cleavage of matrix macromolecules, including type II collagen and the cartilage proteoglycan aggrecan. Cleavage of the triple-helix of type II collagen in particular is known to be mediated by collagenases belonging to the MMP family (Mort, J. S. and Poole, A, R, (2001) supra; Billinghurst, R. C. et al. (1997) supra. Four of the known human collagenases, interstitial collagenase (or collagenase 1; MMP-1), neutrophil collagenase (or collagenase 2; MMP-8), collagenase 3 (MMP-13) and collagenase-4 (Membrane type 1-MMP or MMP-14), have each been shown to first cleave triple-helical type II collagen between residues 775 (glycine) and 776 (leucine) (Mort, J. S. and Poole, A. R. (2001) supra). Each of these collagenases produces a characteristic large TC^{A} (¾) and smaller TC^{B} (¼) cleavage product from the constituent α-chains of type II collagen. See WO 94/14070 and Billinghurst, R. C. et al. (1997) supra. The protease are regulated by specific inhibitors (Mort, J. S. and Poole, A. R. (2001) supra) which are often down-regulated in arthritic cartilages. See Poole, A. R., Alini, M., and Hollander, A. P. Cellular Biology of Cartilage Degradation. In B. Henderson, J. C. W. Edwards, and E. R. Pettipher (ed.): Mechanisms and Models in Rheumatoid Arthritis, pp. 163-204, London, Academic Press, 1995.

Other investigators have shown that the protein content and/or messenger RNA (mRNA) expression of many MMPs, are increased in OA cartilage. See Mitchell, P. G. et al., J. Clin. Invest. 97:761-768 (1996); Reboul, P. et al., Arthritis Rheum. 44:73-84 (2001); Shlopov, B. V. et al., Arthritis Rheum. 40:2065-74 (1997); Freemont, A. J. et al., Ann. Rheum. Dis. 56:542-9 (1997); Shlopov, B. V. et al., Arthritis Rheum. 43:195-205 (2000). Although MMPs are thought to be involved in diseases such as OA, there has been no convincing evidence to date that specific products of MMP cleavage of collagen are involved in the direct activation of cells to produce these proteases molecules to digest the extracellular matrix of cartilage. There have been reports describing the activation of macrophages by peptides of type II collagen (Poole, A. R. et al. (1995) supra) and of stimulation of chondrocyte mediated degradation by digests of type II collagen produced by bacterial collagenase (See Jennings, L. et al., Connect. Tissue Res. 42:71-86 (2001).

Cytokines

It is believed that pro-inflammatory cytokines, such as interleukin-1 (IL-1) and tumour necrosis factor α (TNF-α) are involved in the damage to cartilage in OA by triggering the production of MMPs which in turn leads to extracellular matrix breakdown. See Poole, A. R. (2001 a) supra and Poole A. R. (2001b) supra. It is thought that the induction of MMPs is mediated by chondrocytes in an autocrine/paracrine manner. See Borden, P. et al., J. Biol. Chem. 271:23577-81 (1996); Kammermann, J. R. et al., Osteoarthritis Cartilage 4:23-34 (1996); MacNaul, N. K. et al., J. Biol. Chem. 265:17238-17245 (1990); and Goldring, M. B. Arthritis Rheum. 43:1916-1926 (2000).

It has been shown that IL-1 or TNF-α or a combination thereof, can induce expression of several pro-inflammatory factors, including cyclooxygenase 2 (COX-2), inducible nitric oxide synthase (iNOS) and phospholipase A₂, providing further evidence of the role of these cytokines in the inflammation observed in RA. Further evidence of the involvement of these cytokines is provided by the elevation of the amount of IL-1 and TNF-α found in OA synovial fluids, and the upregulation of these genes in OA cartilages See Poole, A. R. (2001b) supra. Furthermore, IL-1 and TNF-α receptors are upregulated in OA cartilage. See Goldring, M. B. supra and Poole, A. R. (2001b) supra.

These observations involving chondrocytes suggest that IL-1 and TNF-α are generated by these cells in increased amounts in OA and may therefore contribute to the pathology.

Chondrocyte Differentiation

Chondrocyte differentiation is an integral feature of skeletal development occurring in endochondral ossification. See Poole, A. R. (2001b) supra. In OA, chondrocytes frequently differentiate and become hypertrophic in the more superficial degenerate extracellular matrix (Goldring, M. B. supra; Poole, A. R. (2001b) supra), where type II collagen damage is more pronounced (Hollander, A. P. et al., (1994a) supra). They exhibit the hypertrophic phenotype characterized by type X collagen expression and synthesis, apoptosis, up regulation of MMP-13 and vascular endothelial cell growth factor. See Poole, A. R. (2001b) supra.

Degradation of Collagen in Disease

Rheumatoid Arthritis

Rheumatoid arthritis (RA) is a chronic inflammatory disease characterized by inflammation of many diarthrodial joints, resulting in progressive destruction of articular and periarticular structures. Damage to type II collagen fibrils is commonly seen in RA, particularly around chondrocytes in the deep zone of articular cartilage next to the subchondral bone, as well as adjacent to pannus tissue. See Dodge, G. R. and Poole, A. R. (1989) supra and Poole, A. R. et al., Acta Orthop. Scand. Suppl. 266:88-91 (1995).

Osteoarthritis (OA)

OA, like RA, is another debilitative condition. It represents a complex of interactive degradative and reparative processes in cartilage and bone with secondary inflammatory changes. It results in a progressive degeneration of diarthrodial joints in particular a loss of articular cartilage, resulting in a loss of joint function. Recent studies have demonstrated that excessive degradation, involving cleavage and denaturation of most particularly (but not exclusively) type II collagen in human articular cartilage is implicated in osteoarthritis. See Hollander, A. P. et al. (1994a) supra; Dodge, G. R. and Poole, A. R. (1989) supra; Hollander, A. P. et al. (1995) supra; Billinghurst, R. C. et al. (1997) supra; Dahlberg, L. et al. (2000) supra; and Wu, W. et al. Arthritis Rheum. 46:2087-2094 (2002).

Primary or idiopathic OA affects interphalangeal joints, and other small joints as well as large joints, such as the hip or knee. The disease may involve one particular joint, or it may be more generalized and involve multiple joints. OA may be genetically transmitted (such as a consequence, for example, of a mutation in the type II collagen COL2A1 gene) and therefore is known as familial OA. OA may develop in patients after traumatic injury or damage to chondrocytes associated with abnormal deposits in the cartilage matrix found in metabolic diseases such as hemochromatosis, ochronosis or alkaptonuria, Wilson's disease, and Gaucher's disease. Idiopathic OA may result from disturbances in cartilage metabolism caused by endocrine disorders (Poole A. R. (2001b) supra). Mineralization of cartilage matrix is also a feature of OA (Poole A. R. (2001b) supra) and is associated with chondrocyte hypertrophy.

Psoriatic Arthritis

Psoriasis is an inflammatory disease involving skin and its proliferation. Some 10-40% of patients develop a chronic inflammatory erosive arthritis closely resembling rheumatoid arthritis in that it involves destruction of articular cartilages.

Ankylosing Spondylitis

Ankylosing spondylitis (AS) is characterized by spinal inflammation associated with degeneration of the intervertebral disc, sacroiliitis, and inflammatory erosive joint disease of the appendicular skeleton in about one quarter of patients. It is also characterized by inflammation of the entheses where type II collagen is present. See Visconti, C. S. et al., Arch. Biochem. Biophys. 329:135-142 (1996)

### SUMMARY OF THE INVENTION

The present invention is based upon the surprising discovery that degradation of collagen is auto-regulated and we have identified peptide fragments of the collagen which are involved in the modulation of the collagen species' own further degradation.

It is an object of the present invention, therefore, to provide products and methods for regulating the degradation of collagen, including type II collagen and modulating both cell differentiation and the degradation of collagen in vitro and in vivo. It is also another object of the invention to provide variants, inhibitors, antibodies, and mimetics of these peptide fragments and inhibitors of the proteases producing these peptide fragments which are capable of modifying the degradation of collagen so as to reduce the pathological effects of increased collagen destruction. It is further an object of the invention to provide methods for screening peptide fragments of the invention useful for diagnostic purposes.

In accordance with these and other objects, the invention provides an isolated or purified peptide comprising an amino acid sequence selected from the group consisting of:

(a) PRGPPGPPGKPGDDGEAGKPGKSGERGPPGPQGARGFPGTPGLPGVKGH RGYPGLDGAKGEAGAPGVKGESGSPGQNGSPGGPM (CB12);

(b) GPRGPPGPPGKPGDDGEAGKPGKSGERGPPG (CB12-I);

(c) ERGPPGPQGARGFPGTPGLPGVK (CB12-II);

(d) GLPGVKGHRGYPGLDGAKGEAGAPG (CB12-III);

(e) GEAGAPGVKGESGSPGQNGSPGPM (CB12-IV);

(f) GERGPPGPQGARGFP*GTP*GLP*GVK wherein the * denotes sites of hydroxylation. (Pro6);

(g) GERGPP*GPQGARGFPGTP*GLP*GVK wherein the * denotes sites of hydroxylation. (Pro15);

(h) GERGPP*GPQGARGFP*GTPGLP*GVK wherein the * denotes sites of hydroxylation. (Pro18); and

(i) GERGPP*GPQGARGFP*GTP*GLPGVK wherein the * denotes sites of hydroxylation. (Pro21) or a fragment or conservatively substituted variant thereof, wherein said peptide is effective in altering the rate of degradation of type II collagen or the rate of chondrocyte hypertrophy. In addition, the invention provides a peptide fragment consisting essentially of an amino acid sequence denoted as an overlapping peptide: GKSGERGPPG.

In accordance with one embodiment, the above-mentioned purified or isolated peptides or peptide fragments can be further modified by hydroxylation at one or more of the proline or lysine residues of the peptides. The hydroxylated proline or lysine residues maybe located within the sequence Gly-X-Pro or Gly-X-Lys, wherein X indicates any amino acid.

In accordance with yet another embodiment, one to five amino acids of the peptides of the present invention have been replaced using conservative substitutions and wherein these peptides are effective in altering the rate of degradation of type II collagen or the rate of chondrocyte hypertrophy.

Furthermore, the present invention encompasses peptides that have at least 80% homology to the above-mentioned peptides wherein these peptide are effective in altering the rate of degradation of type II collagen or the rate of chondrocyte hypertrophy.

In one aspect of the present invention, the peptides can be in form of a peptide dimer or trimer selected from the group of peptides as mentioned above. The peptide dimer consists of two peptides chosen from the peptides of the present invention. The peptide can further be a homodimer or a heterodimer. Similarly, the peptide trimer consists of three peptides wherein each peptide is selected from the group of peptides as discussed above. The peptide trimer can be a homotrimer or a heterotrimer.

In yet another aspect of the instant invention, there is a provision for variants, inhibitors, antibodies, and mimetics of the peptides of the present invention. Pharmaceutical compositions that comprise a pharmaceutically effective carrier and at least one of the inhibitors of these peptides are also provided. The pharmaceutical compositions, in turn, may reduce collagen matrix turnover in mammals, preferably humans.

A further aspect of the present invention, there is also provision for a method of regulating collagen turnover that comprises the administration of a pharmaceutically effective amount of the above-mentioned pharmaceutical compositions. Such pharmaceutical compositions may reduce degradation of one or more collagen proteins.

The present invention also embodies a method of identifying a peptide mimetic of the peptide fragments of collagen capable of decreasing the degradation of the collagen in a biological sample that comprise screening peptide fragments of collagen, and variants thereof for the ability of the peptide fragments to bind preferentially to a specific receptor of the naturally produced peptide fragments but has a lesser ability to activate the matrix degradation pathway. The specific receptors can be anti-integrin receptors. The activation of the matrix degradation pathway induces the expression of genes selected from the group consisting of COLX, MMP-9, TGF-B1, IHH, MMP-13, CBFA1, SOX 9, bFGF, pTHrP, caspase-3, MT1-MMP, IL-1B, and MMP-1.

Yet another aspect of the present invention, the biological sample can be a biological fluid selected from the group consisting of synovial fluid, serum and urine.

A further embodied feature of the present invention includes isolated or purified antibodies that may specifically bind to an epitope of the above-mentioned peptides or antigenic fragments thereof. The antibodies can be a monoclonal or a polyclonal antibodies effective in inhibiting the activity of these peptides. These antibodies can be used to identify inhibitors of the generation of the inventive peptides. The antibodies of the present invention can be used to identify a subject at risk for rapid or slow progression of a disease, responding to therapy designed to arrest cartilage degradation or at risk for a disease by showing of early pre-clinical changes prior to clinical presentation of the disease. The disease is selected from the group consisting of osteoarthritis, rheumatoid arthritis, post-traumatic osteoarthritis, idiopathic osteoarthritis, and eye disease. These antibodies can also be employed in a method of diagnosing a disease selected from the group consisting of osteoarthritis, rheumatoid arthritis, post-traumatic osteoarthritis, idiopathic osteoarthritis, and eye disease. In addition, they can be used to detect the release of type II collagen degradation products in body fluids selected from the group consisting of tissue extracts, serum, synovial fluid, and urine. Furthermore, they can be utilized in a method of inhibiting chondrocyte hypertrophy in a subject comprising administering to the subject a pharmaceutically effective amount of these antibodies, whereby said hypertrophy is inhibited.

There is also a provided a method of screening for a compound capable of inhibiting collagen breakdown comprising:

(a) incubating the test compound in vitro with an extract containing collagen;

(b) adding a compound known to increase degradation of collagen; and

(c) selecting the compound capable of decreasing the degradation of collagen as compared with the known compound alone.

Other features and advantages of the present invention will become apparent from the following detailed description. It should be understood, however, that the detailed description and illustrated examples, while indicating preferred embodiments of the invention are given by way of illustration only, since various changes and modifications within the spirit of the invention will become apparent to those skilled in the art.

### BRIEF DESCRIPTION OF THE DRAWINGS

The objects and features of the invention can be better understood with reference to the following detailed description and drawings.

FIG. 1 is a table of one embodiment of the invention showing the amino acid sequences of peptide sub-fragments, CB12-1, CB12-II, CB12-III and CB12-IV, of the type II collagen CB12 peptide. CB12 is most capable of enhancing type II collagen degradation. Peptides which contain hydroxylated proline are shown by the presence of the asterisk.

FIG. 2 shows time courses of changes in total collagenase-cleaved type II collagen content in pellet cultures of adult bovine articular chondrocytes treated with and without cyanogen bromide (CNBr) fragments of type II collagen. CNBr fragments at 1 μM (▪) and 10 μM (▴) were added to serum-free media from day 0. Control cultures ( ) were without any additives. In some cultures 10 nM RS 102,481, an inhibitor of collagenase-3 (MMP-13), was added with the 10 μM CNBr fragments (◯). Significant differences were observed for CNBr fragments and for the inhibitor compared to no inhibitor plus fragments. The measurement of collagen II cleavage by immunoassay is described in Billinghurst, R. C. et al. (1997) supra.

FIG. 3 is graph showing one embodiment of the invention with time courses of change showing a lack of effect of CNBr fragments on the C-propeptide of type II procollagen (CPII, a measure of type II procollagen synthesis, see Nelson, F. et al. J. Clin. Invest. 102:2115-25 (1998)) in cell pellets of isolated chrondrocytes from bovine adult articular cartilage treated with and without CNBr fragments of type II collagen at 1 μM (▪), 10 μM (▴), and control cultures (●) with no additives.

FIG. 4 is a graph of one embodiment of the invention demonstrating no effect on time course changes in proteoglycan (sulfated glycosaminoglycan, GAG) content in cell pellets, media and DNA content with and without treatment with CNBr fragments of type II collagen. 1 μM CNBr fragments (▪), 10 μM CNBr fragments (▴). Control cultures (●).

FIG. 5 is a series of graphs of one embodiment of the invention showing: FIG. 5A shows time courses of changes in collagenase-cleaved type II collagen content in both cartilage and medium with treatment with denatured type II collagen and CB12 in bovine articular cartilage explant culture. Denatured type II collagen and CB12 were added at 0.1 (▪) and 1 μM (▴) from day 0. Control cultures (●) were without any additives. Values are mean±SD for 4 determinations. One-way ANOVA confirmed a significant effect of CB12 on resulting COL2-3/4C content in both cartilage and medium on day 12. FIG. 5B shows time courses of changes in GAG content in cartilage and GAG release into medium with treatment with denatured type II collagen and CB12 in bovine explant culture. Denatured type II collagen and CB12 were added at 0.1 (▪) and 1 μM (▴) from day 0. Control cultures (●) were without any additives. Values are mean±SD for 4 determinations. One-way ANOVA revealed no significant effects on GAG release into medium nor on GAG content in cartilage.

FIG. 6 is a graph of one embodiment of the invention showing time courses of increases in (FIG. 6A) collagenase-cleaved type II collagen in both cartilage and medium with treatment with various peptide fragments of the invention in explant cultures of isolated chondrocytes from adult bovine articular cartilage. Peptide fragments CB12-I, II, III, and IV were added at 1 μM (▪) and 10 μM (▴). Control cultures (●) were without any additives. Peptide CB12-II was most active. FIG. 6B demonstrates time course changes in GAG content in cartilage and GAG release into medium with treatment of CB-12 derived synthetic peptides in explant cultures. There were no consistent differences where CB12-I, II, III and IV were added at 1 μM (▪) and 10 μM (▴). Control cultures (θ) had no additives.

FIG. 7A is a graph of one embodiment of the invention showing a dose dependent induction over 16 days by the peptide fragment CB12-II (SP) of type II collagen cleavage by collagenase in normal human adult articular cartilage explants (cartilage and medium combined) (57 year- old female). FIG. 7B is a bar graph of one embodiment of the invention showing peptide fragment CB12-II induction over 16 days of type II collagen cleavage by collagenase in a dose-dependent manner in normal adult human articular cartilage explants (cartilage and media combined) from a 57-year old female and a 67-year old male. Activity was seen at 51M and 10 μM in both cases and at 1 μM in the 57 year old.

FIG. 8 is a bar graph of one embodiment of the invention showing the important effect of differences in proline hydroxylation on activity of CB12-II (SP) induction on type II collagen cleavage by collagenase in normal human articular cartilage in explant culture. Different hydroxylated peptides are listed in FIG. 1.

FIG. 9, in one embodiment of the invention, demonstrates that the induction of MMP-1 (FIG. 9A) and MMP-13 (FIG. 9B) released into medium from isolated adult human articular chondrocytes incubated with CB12-II (SP) at 50 μM, USP at 50 μM, and TNF-α at 50 ng/ml for 48 h, as measured by ELISA.

FIG. 10, in one embodiment of the invention, demonstrates that peptide CB12-II (SP) did not induce proteoglycan degradation [release into medium with depletion of GAG in cartilage of normal human adult articular cartilage explants]. Cumulative proteoglycan (mainly aggrecan) released into medium was calculated by summation of proteoglycan contents in medium at each medium change of every four days. Cartilage explants were maintained for up to 16 days cultured with or without CB12-II at 10 μM. Sulfated glycosaminoglycan in medium was assayed by dimethylmethylene blue (DMMB) method. Data are expressed as μg GAG release per wet weight of cartilage.

FIG. 11, in one embodiment of the invention demonstrates that expression of integrins on human chondrocytes freshly isolated from normal articular cartilage. After isolating chondrocytes by overnight cartilage digestion using trypsin and collagenase, cells were resuspended and recovered for 2 h. Thereafter, cell suspension was incubated with fluoresceinated anti-integrin antibodies the binding of which were determined cytometrically by FACSCAN. β and α integrins were most strongly expressed. β1, α2, α5 and α2β1 and α5β1.

FIG. 12, in one embodiment of the invention, demonstrates and example of experiment showing an attachment of isolated chondrocytes to CB12-II (FIG. 12A), a non-specific peptide (CB12-IV; (FIG. 12B)) or human fibronectin (FIG. 12C). Blocking effects of anti-integrin antibodies and control immunoglobulin (IgG) were compared to the control (no immunoglobulin). Attachment was measured as total cellular hexosaminidase activity as shown. Antibodies to α5β1 integrin blocked adhesion to CB12-II (FIG. 12A) and fibronectin (FIG. 12C). Antibodies to α2β2 and cx integrins also blocked adhesions in some patients (data not shown). Antibodies had no effect on adhesion to CB12-IV (FIG. 12B).

### DETAILED DESCRIPTION OF THE INVENTION

Definitions

The term “antibody” as used herein includes antibodies that react with one or more of the peptide fragments in CB12 as well as antibodies to proteinases that create one or more of the peptide fragments of the invention. The term “antibodies” is also intended to include parts thereof such as Fab, Fv fragments as well as antibodies that react with the overlapping regions of one or more of the peptide fragments of the invention and recombinantly produced fragments and fusion products of antibody fragments (including multivalent and/or multi-specific). The term “antibodies” is also intended to include antibodies to receptors specific for one or more of the peptide fragments of the invention. Antibodies can be fragmented using conventional techniques and the fragments screened for utility in the same manner as described above. Antibodies may be used either for screening for diagnostic purposes or in order to identify additional peptide fragments, mimetics, variants and inhibitors of the invention. Antibodies can also be used to identify proteases which are responsible for creation of the peptide fragments of the invention in vivo.

By the term “collagen” or “species of collagen” is meant any of the 25 different collagen a chains which occur in at least 19 different collagen types, designated type I through type XIX (Mayne R. (2001) supra; Kiety et al., 1993, ref. quoted earlier). By the term “collagen peptide” is meant any fragments of collagen a chains incorporating the active peptide fragments earlier described in this invention.

As used herein, the term “degradative pathway” or “cascade of events” or “degradation cascade” is meant to include both events prior to the creation of the peptide fragments of the invention, including creation of α chain fragments incorporating the sequences of said peptide fragments by specific or non specific proteases as well as subsequent events triggered by the release of peptide fragments of the invention, as would be understood by a person skilled in the art. More specifically, events subsequent to release of the peptide fragment of the invention can include but are not limited to further cleavage of collagen and associated proteoglycans by other collagenases and metalloproteinases.

In addition, the cascade of events triggered by the release of peptide fragments of the invention may include activation of cytokines such as IL-1, TNF-α, IGF-1, TGF-β and the like, so as to upregulate or downregulate same and activation or inhibition of other cascade events such as signaling pathways influencing expression of genes encoding for proinflammatory cytokines and degradative proteases and their inhibitors.

The term “dosing”, as used herein, refers to the administration of a substance (e.g. peptide fragments, as well as variants, inhibitors, and mimetics of the peptide fragments as disclosed herein) to achieve a therapeutic objective (e.g. the treatment of a collagen degradation associated disorder).

By the terms “functionally equivalent variant” or “variant” is meant minor modifications to the peptides described herein, and may include replacement of one or more amino acids with one or more amino acid substitutions, insertions, and/or deletions. Amino acid substitutions may be of a conserved nature or may be non-conserved. Conserved amino acid substitutions may involve replacing one or more amino acids of the proteins of the invention with amino acids of similar charge, size, and/or hydrophobicity characteristics. Non-conservative substitutions involve replacing one or more amino acids which possess dissimilar charge, size, and/or hydrophobicity characteristics. Variants also include post translational modifications to the peptide fragments, including enzymatic and non-enzymatic modifications, including glycosylation, glycation, hydroxylation and the like. The term “variant” also encompasses minor variations as described above to the mimetics and inhibitors of the invention. By making such modifications a peptide may bind to a receptor but not activate it, thereby not allowing naturally derived peptides from stimulating cartilage degradation. Alternatively, a variant of this peptide may have more capacity to induce cartilage degradation. As used herein, by the term “hydroxylation” is meant the modification of one or more amino acids within a peptide fragment. More particularly, hydroxylation can refer to hydroxylation of proline residues at one or more positions and even more particularly can include hydroxylation of proline within the amino acid sequence Gly-X-Y wherein X is any amino acid and Y is proline.

As used herein, by the term “inhibitor” or “inhibitor molecule” is meant a substance or a group of substances having the ability to alter or prevent the activation of the specific receptor by the peptide fragment or fragments of the invention. By “inhibitor” is also intended a substance or a group of substances having the ability to alter and/or prevent the binding of the peptide fragment or fragments to the specific receptor so as to either inhibit or minimize the interaction between the peptide fragments and the specific receptor(s). By the term “inhibitor” or “inhibitor molecule” is also meant the ability to competitively inhibit the interaction of the wild type peptide fragments with its specific receptor as determined by a competitive inhibition assay as described herein or so as to allow binding of the peptide fragment to the receptor but prevent or minimize the ability of the peptide fragment to activate the degradation pathway. By the term “inhibitor” or “inhibitor molecule” is also meant substance or a group of substances capable of interacting with one or more proteases so as to inhibit the creation or level of active peptide fragments.

As used herein, by the term “ligand” is meant one or more of the peptide fragments of the invention or one or more epitopes of the peptide fragments of the invention which bind to one or more receptors on the surface of the chondrocyte or other cell surface involved in the activation of the degradation pathway and can activate the cascade of events which lead to the degradation of collagen .

By the term “mimetic” is meant a substance that mimics one or more of a combination of peptides of this invention so as to activate, modulate, inhibit or suppress the auto regulation of the degradation of the protein from which the peptide is derived. A mimetic may also represent a substance that mimics the activity of the peptide inducing the degradation of cartilage matrix.

As used herein, “modulation of degradation” is meant the ability to increase or decrease the rate of degradation of collagen or increase or decrease the accumulation of one or more of the products of collagen degradation and may include modulation of the rate of degradation of collagen or of the pattern of degradation.

By the term “peptide” or “peptide fragment” is meant any short chain of amino acids (peptides and oligopeptides) comprising amino acids joined to each other by peptide bonds or by modified peptide bonds, i.e. peptides which have the ability to activate the degradation pathway or cascade of events. Peptides according to the invention will generally be between 5 to 100 amino acids in length preferably, between 10-90 amino acids in length, more preferably between 20-80 amino acids in length. Peptide fragments of the invention modulate the cascade of events which lead to the degradation of the collagen molecule. The amino acid sequences of the peptide fragments and the nucleotide molecules encoding these amino acid sequences are also contemplated within this invention.

As used herein “peptide fragment precursor” is meant the α chain or fragment thereof containing the peptides of the invention and can include the full length α chain of collagen molecule, or a partially degraded collagen molecule which with one or more cleavage events that leads to the resulting peptide fragment or a fragment that includes it. The peptide can also represent sequence in the denatured molecule which has not been released from that α chain.

By the term “specific receptor” for the peptide fragment is meant a receptor for which the naturally occurring (in health and/or disease) peptide fragment exhibits a high binding affinity and which under conditions of interaction of said wild type peptide fragment with said specific receptor leads to the activation of the cascade of events which result in a modulation or degradation of the full length protein from which the naturally produced peptide fragment is derived. Specific receptors can include but are not limited to type II collagen receptors, such as integrin and the integrin receptor subtypes.

As used herein, by the term “therapeutic agent” or “agent” is meant a compound that can be used to modulate the degradation of collagen in vivo in mammals, including humans, in a manner which enables treatment of one or more disease states which result directly or indirectly from collagen degradation. A therapeutic agent according to the invention also refers to peptide variants, peptide fragments, mimetics or inhibitors or antibodies as disclosed herein. The invention provides for a “therapeutic agent” that 1) prevents the onset of disease wherein a characteristic of the disease is the degradation of collagen; 2) reduce, delay, or eliminate symptoms such as pain, swelling, weakness and will prevent loss of functional ability in the afflicted joints of said disease; 3) reduces, delays, or eliminates cartilage degeneration. It should be noted that one may reduce cartilage degeneration without necessarily influencing pain and inflammation.

As used herein, by the term “naturally produced peptide fragments” is meant the one or more degradation products of the species of collagen, which exist naturally in mammals, more specifically humans, which occur as a result of the degradation of the species of collagen as it occurs within a normal individual wherein said naturally produced peptide fragment when produced in sufficient quantity results in a measurable variation in the rate or amount of degradation of the species of collagen from which the naturally produced fragment is derived. For greater certainty, the term “naturally produced peptide fragment” is also meant to include synthetic peptide or CNBr cleaved peptide fragments which have the same sequence and produce a significant increase or decrease in the degradation of the full length collagen. Such “naturally produced peptide fragments” are identified by their ability to increase or decrease the degradation of full length collagen in vitro or in vivo.

Peptide Fragments of the Invention

Peptide fragments of the invention may include peptides fragments based on the structure of a species of collagen wherein said fragment is capable of modulating the degradation of the collagen species both in vitro and in vivo. Peptide fragments of the invention may either activate the degradation of the said species of collagen or inhibit the degradation of the said species of collagen. Those that inhibit may occur naturally or are produced artificially using an approach such as the kind described above.

Peptide fragments can be synthesized using an amino acid synthesizer or may be purified using techniques known to one skilled in the art. Useful peptides in accordance with the invention are identified by incubating said peptides with a chondrocyte culture or explant culture of cartilage with the said peptide in the medium and monitoring said sample for an alteration in the amount of degradation of collagen in said sample. In the case of type II collagen, degradation products can be identified using an antibody specific for the collagenase cleavage neoepitope seen by the antibody COL 2-3/4C as disclosed herein and in PCT/CA93/00522 which is incorporated herein by reference. Similarly, it would be understood by a person skilled in the art that one could utilize similar methods to monitor increased degradation of other collagen species using the approach described in Hollander, A. P. et al., J. Cell. Biochem. 28:15-21 (1994b) and Billinghurst, R. C. et al. (1997) supra. For example, isolated chondrocytes isolated from bovine and human articular cartilage in pellet cultures, or explant cultures of mature articular cartilage can β be used and monitored for degradation products of the collagen species such as hydroxyproline release into culture medium. Peptide fragments can also be incubated with cultures from chondrocytes such as those involved in endochondral ossification (bone formation) such as are formed in the physes or fracture callous of growth plates. In addition peptide fragments useful in accordance with this invention can be incubated with other collagens and matrices such as skin, lung, ligaments and tendons and monitored for degradation products of the collagen species present in these samples.

Peptide fragments, be they naturally occurring or diseased variants, which activate the degradation cascade and in particular which increase or decrease the rate or amount of degradation, can be identified by assays designed to identify changes in the amount of peptide fragments or changes to the rate of degradation product accumulation. Peptide fragments capable of decreasing the rate or amount of degradation can be identified by identifying those peptide fragments that stimulate or down regulate the synthesis of gene products known to be involved in the degradation cascade. For example, with respect to the degradation cascade of type II collagen, in arthritis it has been shown that IL-1, TNF-α, MMP-1 and MMP-13 genes are upregulated as further described herein. So any peptide fragments which can decrease gene and/or protein expression of these examples would be of use in regulating cartilage degradation.

Peptide fragments may also be modified so as to alter the ability of the peptide to modulate degradation of the collagen (either enhance or decrease degradation), for example, joining peptides so as to form homodimers or heterodimers, homotrimers, heterotrimers and the like. Similarly, other modifications including both enzymatic and non-enzymatic modifications including hydroxylation, glycosylation, glycation and other similar modifications known to persons skilled in the art can be utilized which can modulate the activity of the peptide.

In particular, hydroxylation at various residues can alter the activity of the peptide significantly and may vary as the individual ages, or according to the disease with varying degrees of disease where synthesis is increased and altered post-translational modification may occur. Hydroxylation can occur at various amino acids including proline and lysine residues. More particularly, hydroxylation occurs at proline and lysine residues and even more particularly proline and lysine residues within the Gly-X-Y triplet repeat in the helical domains of α chains, wherein X is any amino acid and Y is proline or lysine. This has been demonstrated to be an important factor in the ability of the peptide fragments of type II collagen to modulate degradation.

Variants

Variants of peptide fragments of the invention include insertions, deletions, conserved amino acid substitutions and non-conserved amino acid substitutions wherein the variant is capable of modulating the degradation of the wild type peptide fragment. One or more amino acid insertions or deletions may be introduced into peptide fragments of the invention. Amino acid insertions may consist of a single amino acid residue or sequential amino acid insertions ranging from 1-100, more particularly 1-50, more particularly 1-10 amino acids in length. For example, amino acid insertions may be used so as to maintain the secondary or tertiary structure of the peptide fragment and thus maintain ability of the peptide fragment to bind to target receptors while preventing the peptide fragment from activating the degradation cascade. The opposite may occur with these peptide variants, such that they may be used in vivo to inhibit the activity of a wild type peptide fragment which increases degradation of the full length collagen protein.

Deletions may consist of single amino acid deletions or sequential amino acid deletions ranging from approximately 1-50 amino acids, preferably 1-10 amino acids, more preferably 1-5 amino acids and most preferably less than 5 amino acids. For example, amino acid deletions may be used so as to maintain the secondary or tertiary structure of the peptide fragments and thus maintain the ability of the peptide fragment to bind to specific receptors as described above.

Variants of the peptides of the invention peptide fragments are most conveniently prepared by chemical synthesis. The invention also contemplates isoforms of the peptide fragments of the invention. An isoform contains the same number and kinds of amino acids as a protein of the invention, but the isoform has a different molecular structure. The isoforms contemplated by the present invention include cyclic peptides. Isoforms may have the ability to bind to the specific receptor and/or preferentially or competitively bind to the specific receptor as compared to the wild type peptide fragments but demonstrate a lesser ability to activate the degradation pathway. In addition, isoforms may act like the naturally produced peptide fragment but have a unique metabolic pathway allowing for increased or decreased ability to clear the peptide fragment from the system.

The peptide variants of the invention also include homologs of the amino acid sequences of the invention and/or truncations thereof as described herein. Such homologs include peptides with an amino acid sequence having at least 70% preferably 75% more preferably 80%, most preferably 90% identity with the peptide fragments of the invention.

Peptide Mimetics

The peptide mimetics of the invention should ideally be able to bind preferentially to the specific receptor of the naturally produced peptide fragments but should demonstrate a lesser ability to activate the degradation pathway or cascade of events. By this it is meant that the peptide mimetic should ideally bind to a specific receptor with similar or greater affinity as compared with the wild type peptide fragments, but prevents activation of said specific receptor, or demonstrate a lesser ability to activate the degradation pathway. The peptide mimetic also should not, to any significant degree, bind to molecules that the naturally occurring breakdown products do not bind to. Of course, by careful screening, peptide mimetics according to the invention may be chosen to possess selected properties of the wild type breakdown products, to suit the application of choice (for example, binding to a subset of receptor targets bound by naturally occurring degradation products).

In order to be useful in providing potential lead drug compounds, peptide mimetics of the invention should bind to the target molecule with an affinity of at least 1 mM, preferably 1 μM, more preferably at least 50 nM, most preferably at least 1 nM. Peptide mimetics may also contain amino acids other than the 20 nucleotide-encoded amino acids, wherein said amino acids are modified either by natural processes, such as by post-translational processing, or by chemical modification or chemicals synthesis techniques which are well known in the art. The inclusion of such amino acids may resolve a problem that is inherent in the pharmaceutical use of the naturally occurring peptides, which are generally degraded and/or eliminated rapidly in vivo.

Examples of known modifications which may commonly be present in peptides of the present invention are glycosylation, glycation, hydroxylation, lipid attachment, sulphation, gamma-carboxylation of glutamic acid residues, and ADP-ribosylation, for instance. Other potential modifications include acetylation, acylation, ADP-ribosylation, amidation, covalent attachment of flavin, covalent attachment of a haeme moiety, covalent attachment of a nucleotide or nucleotide derivative, covalent attachment of a lipid or lipid derivative, covalent attachment of phosphotidylinositol, cross-linking, cyclization, disulphide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulphation, transfer-RNA mediated addition of amino acids to proteins such as arginylation and ubiquitination.

Modifications can occur anywhere in the peptide, including in the peptide backbone, the amino acid side-chains and the amino or carboxyl termini. In fact, blockage of the amino or carboxyl group in a peptide, or both, by a covalent modification, is common in naturally-occurring synthetic peptides and such modifications may also be present in peptides of the present invention.

A particularly preferred peptide mimetic according to the present invention is a mimetic of a peptide fragments of collagen, more particularly type II collagen wherein said peptide mimetic can modulate the degradation of one or more species of collagen molecule.

In one embodiment of the present invention, mimetics of the peptide fragments of type II collagen particularly CB12, more particularly a mimetic of the peptide fragment selected from the group consisting of: CB12-I, CB12-II, CB12-III, CB12-IV, SP1, SP2, SP3, Pro6, Pro15, Pro18 and Pro21. Even more particularly, a mimetic of the peptide fragment selected from the group wherein such mimetics prevent the increased degradation of type II collagen observed in disease states wherein said disease is caused, either directly or indirectly by degradation of collagen and includes: osteoarthritis (OA), rheumatoid arthritis (RA), juvenile arthritis (JA) post-traumatic osteoarthritis (post-traumatic OA), post-traumatic and idiopathic osteoarthritis, psoriatic arthritis, ankylosing spondylitis, eye disease involving collagens, lung and skin disease and the like.

Consequently, drugs that are able to interfere with the activity of the peptide fragments of the invention will help reduce the degradation of the full length protein from which the peptide fragments are derived and as such will render disease states such as RA, juvenile A, post-traumatic OA and idiopathic OA, psoriatic arthritis, ankylosing spondylitis, eye disease and the like amenable to pharmaceutical control.

Inhibitors

Inhibitor molecules in accordance with the invention are molecules which can inhibit either the creation of the peptide fragments by blocking or preventing protease interaction with the peptide fragment precursors or molecules preventing fragment generation thereby preventing which can prevent the activation of the degradation cascade initiated by the peptide fragments. More particularly, inhibitors can prevent the activation of the degradation cascade by binding or binding preferentially to the specific receptor sites of the peptide fragments of the invention, for example by binding specifically to the surface sites of the receptor(s). Inhibitor molecules act by binding to the recognition site of the peptide fragment or fragments, but not activating the degradation cascade which leads to the activation of the degradation of the collagen molecule.

Inhibitor molecules may also bind to a site of the receptor which is different from the site recognized by the peptide fragment or fragments and induce conformational changes in the receptor molecule such that the receptor is no longer able to be recognized by its ligand.

Inhibitor molecules in accordance with the invention also includes molecules, such as protease inhibitors, which prevent the release of peptide fragments of the invention which activate the degradation cascade. Such inhibitors can be identified, as would be understood by a person skilled in the art, in one instance by incubating the peptide fragment precursor with one or more proteases identified as capable of releasing the peptide fragment and adding potential inhibitors and monitoring for inhibition of release of said peptide fragment.

Similarly, a person skilled in the art can monitor potential inhibitors for the ability to alter the interaction between the naturally occurring peptide fragment and its specific receptor by one of various techniques known in the art including use of a cell attachment assay or an ELISA assay to monitor for the reduction of the binding as between the naturally occurring peptide fragment and the specific receptor.

In addition, we can also determine potential inhibitors by identifying molecules which allow binding of the peptide fragment to a specific receptor, but prevent activation of the degradation cascade.

Antibodies

Isolated or purified antibodies to the peptide fragments described herein may be readily prepared by one skilled in the art given the disclosure provided herein and can be used for assaying purposes, therapeutic purposes or for diagnostic purposes. Antibodies to specific receptor molecules found to interact with the peptide fragments are also encompassed within the present invention and can be used for therapeutic purposes.

A peptide fragment of the invention or antigenic portion thereof can be used to prepare antibodies specific for the peptide fragment. Antibodies can be prepared which bind a distinct epitope of the peptide fragment or can recognize an epitope created by a combination of peptide fragments, either in overlapping regions or to secondary structure elements of, for example dimmers or trimers of peptide fragments. These antibodies can be used to inhibit the activity of the peptide, may be useful for assays designed to identify inhibitors of the generation of said peptide fragments, or may also be used for diagnostic purposes to monitor disease state and disease progression in a variety of tissue samples.

Conventional methods can be used to prepare the antibodies. For example, by using a peptide of the invention, polyclonal antisera or monoclonal antibodies can be made using standard methods. This invention also contemplates chimeric antibody molecules, known to those skilled in the art.

Antibodies as Diagnostics

The antibodies may be labelled with a detectable marker including various enzymes, fluorescent materials, luminescent materials and radioactive materials as is known to those skilled in the art.

Antibodies reactive against naturally occurring peptide fragments of the invention (e.g., enzyme conjugates or labelled derivatives) may be used to detect these peptide fragments and denatured collagen including the peptide sequence in various samples, such as tissue or body fluid samples. For example they may be used in any known immunoassays and immunological methods which rely on the binding interaction between an antigenic determinant of a protein of the invention and the antibodies. Examples of such assays are radioimmunoassays, Western immunoblotting, enzyme immunoassays (e.g. ELISA), immunofluorescence, immunoprecipitation, latex agglutination, and immunohistochemical tests. Thus, the antibodies may be used to identify or quantify the amount of a naturally occurring peptide fragment of the invention in a sample and thus may be used as a diagnostic indicator of disease state.

A sample may be tested for the presence or absence of degradation peptide fragment by contacting the sample with an antibody specific for an epitope of the peptide fragment which antibody is capable of being detected after it becomes bound to a peptide fragment in the sample, and assaying for antibody bound to a peptide fragment in the sample, or unreacted antibody.

In the method of the immunoassay a predetermined amount of a sample or concentrated sample is mixed with antibody or labelled antibody. The amount of antibody used in the method is dependent upon the labelling agent chosen. The amount of peptide bound to antibody or labelled antibody may then be detected by methods know to those skilled in the art. The sample or antibody may be insolubilized, for example, the sample or antibody can be reacted using known methods with a suitable carrier. Examples of suitable carriers are Sepharose or agarose beads. When an insolubilized sample or antibody is used peptide bound to antibody or unreacted antibody is isolated by washing. For example, when the sample is blotted onto a nitrocellulose membrane, the antibody bound to a peptide of the invention is separated form the unreacted antibody by washing with a buffer, for example, phosphate buffered saline (PBS) with bovine serum albumin (BSA).

When labelled antibody is used, the presence of one or more naturally occurring peptide fragments of the invention can be determined by measuring the amount of labelled antibody bound in the sample. The appropriate method of measuring the labelled material is dependent upon the labelling agent.

When the unlabelled antibody is used in a method of the invention, the presence of one or more peptide fragments of the invention can be determined by measuring the amount of antibody bound to one or more of these peptides using substances that interact specifically with the antibody to cause agglutination or precipitation. In particular, labelled antibody against an antibody specific for a peptide of the invention, can be added to the reaction mixture. The antibody against an antibody specific for a peptide of the invention can be prepared and labelled by conventional procedures known in the art which have been described herein. The antibody against an antibody specific for a peptide of the invention may be a species specific anti-immunoglobulin antibody or monoclonal antibody, for example, goat anti-rabbit antibody may be used to detect rabbit antibody specific for a peptide of the invention.

Assay for Identifying Additional Therapeutic Molecules

The present invention encompasses the means of identifying additional naturally occurring peptide fragments, variants, mimetics and inhibitors, and includes use of a number of assays suitable for detecting and identifying such molecules as would be understood to persons skilled in the art, and are briefly described herein, as well as in more detail below in the Methods and Examples.

Assays can be designed so as to identify additional useful peptide fragments, variants, mimetics and/or inhibitors of the invention for treating a disorder associated with collagen degradation which encompass the steps of: (a) selecting a test compound comprising of one or more of a peptide fragment variant, mimetic, or inhibitor, (b) incubating a culture of chondrocytes or cartilage or an extract thereof with the test compound and with a known compound wherein the known compound has a measurable effect on the degradation of the collagen (c) selecting test compounds which alter the degradation of collagen as compared with incubation with the known compound alone. One skilled in the art would understand that those peptides, variants, mimetics or inhibitors which demonstrate a decrease in the degradation of the collagen as compared with incubation with known compounds above are useful, in accordance with the invention, to treat disorders which have, as a contributory factor of said disorder, the degradation of collagen.

Similarly one can identify useful inhibitors of proteases to prevent an increase in peptide fragments shown to increase degradation of collagen by assays designed to identify and quantify the amount of accumulated peptide fragment. For example, antibodies specific for peptide fragments such as disclosed herein can be utilized to measure the level of peptide fragment released from a peptide fragment precursor upon incubation with and without a potential protease inhibitor.

In addition, assays which are encompassed within the scope of the invention include assays to identify those peptide fragments, variants, mimetics and/or inhibitors which allow the wild type peptide fragment to bind to its specific receptor but prevent activation of the degradation cascade.

As would be understood to a person skilled in the art, candidates can be identified by a combination of an assay to test for binding; such as an ELISA or other similar binding assay, and an assay to determine the ability to activate the degradation cascade. The latter can include, but is not limited, to, assays which measure the increase in gene expression of proteins activated as a result of increase degradation such as MMP1, MMP13, IL-1, TNF-α and other similar disease related genes. Similarly, measurement of the activation of the degradation can include assays to measure levels of phosphorylation and therefore activation of signalling proteins or other similar events known to occur within the cell or activation of the degradation cascade.

Also encompassed herein are competitive inhibition assays for use in identifying peptide fragments, variants, mimetics, or inhibitors, in accordance with the invention, for treating a disorder associated with collagen degradation. Such assays can comprise the steps of screening putative peptide fragments, variants, mimetics, or inhibitors (“test compounds”) for the ability to prevent the wild type peptide fragments binding with the target receptor by: (a) pre-incubating target receptor with antibody specific for the receptor, (b) incubating the receptor-antibody complex with test compound (c) removing non specifically bound test compound and (d) identifying test compounds which are able to preferentially bind the receptor as compared with the antibody to the receptor. Other similar competitive inhibition assays can also be utilized, as would be understood by a person skilled in the art.

Therapeutic Compositions and Administration

The peptides, variants, mimetics, inhibitors and antibodies useful in accordance with the invention can be incorporated into pharmaceutical compositions suitable for administration to a subject for the methods described herein, e.g., biweekly, subcutaneous dosing. Typically, the pharmaceutical composition comprises one or more of the above compounds and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible and are suitable for administration to a subject for the methods described herein. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the peptide, variant, mimetic, inhibitor and the like.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, intramuscular). In a preferred embodiment, the peptide, variant, mimetic or inhibitor is administered by intravenous infusion or injection. In another preferred embodiment, the peptide, variant, mimetic or inhibitor is administered by intramuscular injection. In a particularly preferred embodiment, the peptide, variant, mimetic or inhibitor is administered by subcutaneous injection (e.g., a biweekly, subcutaneous injection).

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e. peptide, variant, mimetic or inhibitor) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

One or more of the peptide fragments, variants, mimetics, inhibitors or antibodies, can be administered by a variety of methods known in the art, although for many therapeutic applications, the preferred route/mode of administration is subcutaneous injection. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyethylene glycol (PEG), polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a peptide, variant, mimetic, inhibitor or antibodies, may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, a peptide, variant, mimetic, inhibitor or antibody of the invention is coformulated with and/or coadministered with one or more additional therapeutic agents. For example, an anti-integrin antibody or antibody portion of the invention may be coformulated and/or coadministered with one or more additional peptide, variant, inhibitor, mimetic or the like that bind other targets. Furthermore, one or more peptide fragments, inhibitors, variants, mimetics or antibodies of the invention may be used in combination with two or more of the foregoing therapeutic agents. Such combination therapies may advantageously utilize lower dosages of the administered therapeutic agents, thus avoiding possible toxicities or complications associated with the various monotherapies.

Methods

Purification of Type II Collagen and Generation of Peptide Fragments

Type II collagen was purified from fetal bovine epiphyseal cartilage by pepsin digestion and differential salt precipitation using the method of Miller as described by Dodge, G. R. and Poole, A. R. (1989) supra. CNBr fragments of bovine type II collagen were prepared as described by Dodge, G. R. and Poole, A. R. (1989) supra. Peptide fragments of interest were separated out of the pool of CNBr fragments using high performance liquid chromatography and the identities and composition of the peptides were determined by amino acid sequence analysis.

Isolation of Bovine and Human Articular Chondrocytes and Pellet Cultures

Adult bovine and human articular cartilage was obtained from metacarpophalangeal joints shortly (within 3 hours) after slaughter and from autopsy within 18 hr of death, respectively. Chondroctyes were released from freshly dissected articular cartilage specimens by sequential enzymatic digestion at 37° C. with trypsin and bacterial collagenase as previously described (Aimes, R. T., 1995). The isolated chondroctyes were resuspended at a density of 2×10⁶ cells/ml in Dulbecco's modified Eagle's medium (DMEM; Gibco) containing 50 μg/ml ascorbic acid, 0.1 mg/ml bovine serum albumin (Sigma), and a solution of 5.0 μg/ml insulin, 5.0 μg/ml transferring and 5.0 ng/ml sodium selenite (I. T. S.; Boehringer Mannheim). Following transfer of 1-ml aliquots of the cell suspension into 15-ml centrifuge tubes, the cells were centrifuged at 200×g for 5 min to prepare pellet cultures. The resultant cell pellets were cultured at 37° C. in a humidified atmosphere of 5% CO₂/95% air. The medium was replaced every 3-4 days and cultures were maintained up to 20 days.

Where indicated, CNBr fragments or synthetic peptides were freshly added to culture media from the beginning of culture (day 0) at each medium change. To ensure denaturation, CNBr fragments were heated at 50° C. for 20 min before adding to culture media. Our preliminary studies showed no significant difference between cultures treated with and without denatured (50° C. for 20 min) type II collagen (data not shown). Cultures without any additives were used as control. The pellets and conditioned media were harvested and stored at −20° C. Media were changed every four days.

Human femoral chondylar cartilages were obtained at autopsy within 18 hours of death from adult persons with no known history of arthritis and no macroscopic signs of articular degeneration. None of the persons had diabetes or had received chemotherapy prior to autopsy. Adult bovine articular cartilage was obtained from adults/bovine steers and cows from metacarpalphalangeal joints obtained at the abbatoir immediately after slaughter. All cartilage specimens were removed immediately to the laboratory under sterile conditions.

Cartilage Preparation For Explant Culture

The cartilages were prepared as previously described (Dahlberg, L. et al. (2000) supra. In brief, the cartilage samples were washed three times with basal culture medium containing Dulbecco's modified Eagle's medium (DMEM; Gibco BRL, Life Technologies, Grand Island, N.Y.) with 20 mM HEAPS buffer (pH 7.4) (Gibco BRL), 45 mM NaHCO₃, 100 units/ml penicillin, 100 μg/ml streptomycin and 150 μg/ml gentamycin sulphate (Medium A). Full-depth cartilage slices from a single area, around 20 mm×20 mm were cut vertical to the articular surface and then into cubes of approximately 2 mm×2 mm (5-7 cubes were randomly obtained and wet weights about 50-70 mg/well determined).

Explant Culture

The cartilage was assigned to 48 well plate (ca 40 mg/well) and kept in Medium A supplemented with 50 μg/ml ascorbic acid, 0.1 mg/bovine serum albumin, 50 μg/ml insulin, 5.0 μg/ml and 5.0 μg/ml sodium DMEM alone at 37° C. in 95% air/5% CO₂. The cartilage was precultured for 2 days and medium was changed at day 0. Thereafter medium (as described in pellet cultures) was replaced every four days. Various CNBr fragments and/or synthetic peptides were freshly added from day 0 at each medium change. Thereafter, cartilage explants and conditioned media were harvested and stored at −20° C.

Isolation of Normal Human Chondrocytes

Isolated normal human chondrocytes were used for chondrocyte cultures, detection of integrin expression by FACScan, and cell attachment assays. The dissected normal human cartilage obtained at autopsy was cut into small cubes 2-4 mm in size. Chondrocytes were isolated as previously described (34), with some modifications. Briefly, the cubes were washed three times with medium (A). Thereafter, the diced cartilage was digested with 0.1% (wt/vol) trypsin (Sigma) and 0.02% (wt/vol) EDTA (Sigma) for 60 minutes (25 ml/10 g wet weight tissue) at 37° C. After washing the cartilage with Medium A containing 10% heat-inactivated fetal calf serum (FCS) to inhibit the trypsin, the digestion was continued in the same medium with serum (50 ml/10 g wet weight tissue) containing 0.2% (wt/vol) collagenase (type IA; Sigma) for 16 h at 37° C. with gentle agitation on a gyrotary shaker. Undigested cartilage was removed by filtration through a layer of nylon mesh (Cell Strainer; Becton Dickinson Labware, NJ). Cells were washed by centrifugation (10 minutes, 1500 rpm) in Medium A at room temperature. Cell numbers were estimated with a hemocytometer slide, and viability was checked by trypan blue exclusion.

Culturing of Chondrocytes

The isolated chondrocytes were placed at high density (2×10⁵ cells/cm²) in 15-cm tissue culture dishes (Becton Dickinson Labware, Franklin Lakes, N.J.) and cultured in Medium A supplemented with 10% heat-inactivated fetal calf serum (FCS) in a humidified atmosphere of 5% CO2/95% air. When the cells became confluent, they were trypsinized and passaged once, and then placed at high density onto 6-cm or 10-cm cell culture dishes (Corning Inc., Corning, N.Y.) in Medium A with 10% FCS. The serum was withdrawn 24 hours before the experiment, after the cells were washed with PBS to remove traces of serum. The chondrocytes were incubated with SP, IL-113 and/or TNF-α in Medium A for designated periods of time. Only first-passaged chondrocytes were used for cell culture experiments.

Total RNA Extraction and Isolation

Total RNA was isolated from chondrocytes or cartilage explants by the guanidine isothiocyanate procedure according to Chomczynski and Sacchi (Chomczynski, P. et al., Anal. Biochem. 162:156-9 (1987)) with some modifications. Briefly, chondrocytes (1-2×10⁶ cells) or cartilage tissue (200-300 mg) were solubilized in solution D (4 M guanidine isothiocyanate, 20 mM sodium acetate/pH 5.2, 0.1 M 2-mercaptoethanol and 0.5% N-lauroylsarcosine). One volume of isopropanol was added to the mixture and all proteins and nucleic acids were precipitated at −20° C. overnight. After centrifugation at 4° C., the pellet containing the proteins and nucleic acids was digested with 1 mg/ml proteinase K (molecular biology grade; Gibco BRL) at 65° C. for 2h. After digestion, the mixture was then extracted with 1 volume of phenol and 0.1 volume of chloroform/alcohol (49:1). The aqueous phase was recovered after centrifugation at 4° C. and precipitated with 1 volume of isopropanol at −80° C. overnight. After centrifugation, the pellet was washed with 70% ethanol to remove any excess salt. The total RNA pellet was resuspended in diethyl pyrocarbonate-treated (DEPC) water and the amount of total RNA was measured by reading optical density at 260 nm.

Reverse Transcription

Total RNA (1.5 μg) was reverse transcribed using 200 U SuperScript™ II Reverse Transcriptase (Invitrogen) in a 20 μl reaction volume containing 50 mM Tris-HCl (pH 8.3 at room temperature), 75 mM KCl, 3 mM MgCl2, 10 mM dithiothreitol, dNTP mix (dATP, dTTP, dCTP, and dGTP) of 500 μM each, and 25 μg/ml Oligo-(dT)₁₂₋₁₈ primer at 42° C. for 1 h in a thermal cycler. The reaction was terminated by heating the mixture to 70° C. for 15 min.

Polymerase Chain Reaction (PCR)

One microliter of reverse transcribed total RNA was incubated with 2.5 U of AmpliTaq™ DNA polymerase (Perkin Elmer, Branchburg, N.J.) in 50 mM KCl, 1.5 mM MgCl₂, 10 mM Tris-HCl, pH 8.3, 200 μM dNTP mix and 0.5 μmol each of oligonucleotide primers in 50 μl reaction mixture. PCR amplifications were done in a thermal cycler. The PCR protocol was 30 cycles for denaturation at 95° C. for 1 min, for annealing at 50-58° C. for 1 min and for extension at 72° C. for 5 min followed by a 10 min post-extension at 72° C. The primer sets and annealing temperature for each cDNA are listed in TABLE I. PCR product sizes were verified by electrophoresis of samples containing 3 μl of 1 mg/ml ethidium bromide solution in 1.5% agarose gel in 40 mM Tris, 40 mM acetic acid and 1 mM EDTA. The digital images of the gel were analyzed using NIH 1.60 imaging software to evaluate the pixel intensity of the band of the PCR products. The autobackground subtraction was used to control for the background signal. The band intensities were determined to be below saturation. GAPDH was used as reference for gel loading.

Enzyme-Linked Immunosorbent Assay (ELISA) for Detection of MMPs in Conditioned Medium

First-passaged normal human chondrocytes isolated by the method described above and cultured at high density (2×10⁵ cells/cm²) were trypsinized and resuspended in Medium A containing 1% FCS at a density of 10⁶ cells/ml. Then, 200 μl of the cell suspension was each plated onto round-bottomed 96-well culture plate. On the next day after plating, CB12-II at 50 μM, USP at 50 μM, and TNF-α at 50 ng/ml were added to the culture media, and the culture was maintained for 24-48 hours. Conditioned media were collected and subjected to ELISA assay. The ELISA kits (BIOTRAK) for MMP-1 and MMP-13 were purchased from Amersham Pharmacia Biotech (Piscataway, N.J.). Measurement range was 6.25-100 ng/ml for MMP-1 and 0.094-3 ng/ml for MMP-13. Sensitivity for the assay was 1.7 ng/ml for MMP-1 and 0.7 ng/ml for MMP-13.

Integrin Expression on Chondrocyte Surface Detected by FACScan

After overnight isolation of chondrocytes from normal human cartilage, the cells were filtered, washed with Medium A, resuspended in DMEM with 0.1% BSA, 1 mM PMSF, and 10 μg/ml leupeptin, and recovered for 2 hours at 37° C. on a culture plate. The cells were then washed once, resuspended in ice-cold PBS containing 10 mM HEPES, 1% BSA, 1 mM PMSF and 10 μg/ml leupeptin, and 5×10⁵ cells in 500 μl were aliquoted into small tubes. Anti-integrin antibodies (Santa Cruz) were incubated with the cell suspension at 4° C. for 30 min, followed by the addition of FITC-labeled IgG and incubation for an additional 1 hour at 4° C. After washing the chondrocyte suspension twice with ice-cold PBS, cells were fixed with 1% formaldehyde in PBS for 5 min, washed with PBS, and subjected to FACScan analysis. The cell sorting was performed on an EPICSTM (Coulter Electronics, Inc., Miami Lakes, Fla.) flow cytometer equipped with Cicero software for data analysis.

Cell Attachment Assay

Ninety six-well, non-tissue culture plates were coated with a peptide fragment of the invention (CB12-II), a negative control peptide (USP), or human fibronectin in PBS at various concentrations overnight at room temperature in the laminar flow hood. Preliminary studies revealed that binding increased as the number of cells increased in a range of 2.5-10×10⁶ cells/ml. Optimal binding was obtained with concentration of 10 μg/ml for CB12-II. After the plates were washed once with PBS, additional protein binding sites in the plate wells were blocked with 5% heat-denatured BSA in PBS at 37° [please confirm] for 1 hour. Freshly isolated human chondrocytes were recovered for 2 hours as described above, and then added to on substrate-coated plates at the density of 1×10⁷ cells/ml (0.5-2×10⁶ cells/well) and incubated at 37° C. for 1 hour. The chondrocyte suspensions were preincubated with 5 μg/ml anti-integrin blocking antibodies (from Chemicon): al (clone CB12), a2 (clone P1E6), a5, (clone DID6), α1 (clone 6S6), α2 (cloneP4H9), α3 (clone 25EW), α2β1 (clone BHA2.1), and α5β1 (clone JBS5) at 4° C. for 30 min prior to adding cells to the pre-coated wells for 1 hour at 37° C. Unattached cells were removed and the wells were washed gently twice with PBS. Bound cells were then quantitated by measuring total cellular hexosaminidase as described (36, 37). Sixty microliters of 7.5 mM p-nitrophenyl-N-acetyl-β-D-glucosaminide (Sigma) was added in 0.1 M sodium citrate buffer (pH 5.0) containing 0.5% Triton X-100. After a 6-h incubation, 90 μl of 50 mM glycine, 5 mM EDTA, pH 10.4 was added, and the absorbance was read at 405 nm.

Western Immunoblotsfor Kinase Activity

To detect activation of MAP kinases (ERK1/2, p38 MAPK, and JNK1/2), first-passaged chondrocytes plated onto 10-cm culture dishes were incubated with CB12-II (SP), USP, TNF-α, native human type II collagen, human fibronectin, and antibodies to α2β1 and α5β1 for 5-60 min. In some experiments, chondrocytes were precultured with U0126 (inhibitor of phosphorylation of MEK1/2, up-stream kinase molecule of ERK1/2) at 1 μM for 1 h, SB203580 (inhibitor of phosphorylation of p38 MAPK) at 1 μM for 1 h, Herbimycin A (tyrosine kinase inhibitor) at 1 μg/ml for overnight, Wortmannin (PI3 kinase inhibitor) at 100 nM for 1 h, and Cytochalasin D (focal adhesion inhibitor) at 3 μM for 30 or 60 min before adding peptides.

After a variable but designated incubation time, chondrocytes were washed twice with ice cold PBS and lysed in RIPA buffer (10 mM Tris/HCl, pH 7.4, 0.1% SDS, 1% NP-40, 0.1% sodium deoxycholate, 150 mM NaCl, 1 mM EDTA) containing freshly added proteinase and phosphatase inhibitors (1 mM PMSF, 10 μg/ml leupeptin, 1 μg/ml aprotinin, 1 mM Na₃VO₄, 1 mM NaF). Cell lysate was centrifuged and supernatant was collected. Protein concentration was determined using Bradford's method. Equal amounts of protein were separated on 10% SDS-polyacrylamide gels under reducing conditions and were electrotransferred to a nitrocellulose membrane (Bio-Rad). To block nonspecific binding, the membranes were incubated in PBS containing 5% non-fat milk overnight at 4° C. The membranes were then probed with rabbit antibodies against phosphorylated ERK1/2, p38, and JNK1/2 (New England BioLabs). Antibodies were diluted 1:1000 in PBS containing 0.5% non-fat milk and reacted with the membrane for 1 h at room temperature. Blots were washed with PBS containing 0.1% Tween-20 three times, and incubated for 1 h at room temperature with horseradish peroxidase (HRP)-conjugated goat anti-rabbit IgG (New England BioLabs) diluted 1:2000 with PBS-0.1% Tween 20 for ECL system (Amersham). Then, the membranes were washed with PBS-Tween, and the membranes were detected with the addition of a 1:1 dilution of the ECL detection reagents (Amersham) 1 and 2 for 1 min. The solution was removed, and the membranes were wrapped in plastic wrap and exposed to film for various amounts of time. Total protein loading was demonstrated to be equal by detection of total ERK protein by antibody binding.

Extraction and Assay of Collagenase-Cleaved, and Total Type II Collagen Contents

The cell pellets and cartilage explant cultured for various times were digested for the extraction of collagenase-generated neoepitope COL2-3/4Cₛₕₒᵣₜ (Billinghurst, R. C. et al., (1997) supra) and denatured type II collagen epitope COL2-3/4m and total type II collagen (Hollander, A. P. et al. (1994a) supra), as previously described: Briefly, the harvested pellets were incubated overnight with 1.0 mg/ml α-chymotrypsin at 37° C. to cleave and solubilize denatured collagen leaving the above neoepitope and epitope intact. After inhibition of α-chymotrypsin activity with N-tosyl-L-phenelalanine-chloromethyl ketone (Sigma), the samples were centrifuged and the supernatants were removed and boiled for 10 min. The COL2-3/4Cₛₕₒᵣₜ epitope (hereafter referred to as COL2-3/4Cₛₕₒᵣₜ) generated by cleavage of type II collagen by collagenase (Billinghurst, R. C. et al. (1997) supra) and intrachain epitope (COL2-3/4m) exposed in denatured type II collagen (Hollander, A. P. et al. (1994a) supra) were measured in α-chymotrypsin extracts by immunoassay. The COL2-3/4Cₛₕₒᵣₜ epitope is detected in an ELISA assay using a rabbit antibody to the carboxyterminal cleavage neoepitope on the TCA piece generated by primary collagenase cleavage of type II collagen. The release of the COL2-3/4C epitope into media was also measured by immunoassay. Total contents in tissue/cells and media was recorded for COL2-3/⁴Cₛₕₒᵣₜ epitope. The COL2-3/4m epitope was recorded in cells and tissue where it was concentrated. The remaining residues of pellets and explants were digested overnight with 1.0 mg/ml proteinase K at 56° C. to extract the remaining intact type II collagen and then boiled for 10 min to denature the enzyme. Total type II collagen content in the pellets and explant was determined from the collective amount of COL2-3/4m in both the α-chymotrypsin and proteinase K digests.

Radioimmunoassay (RIA) of C-Propeptide of Type II Procollagen (CPII)

In order to show the specificity of the peptide fragments of one embodiment of the invention on synthesis and not degradation, an RIA of C-propeptide of type II procollagen (CPII) was performed. The immunoassay (RIA) for CPII has been described previously and the peptide has been shown to be a marker of type II collagen synthesis (Nelson, F. et al. (1998) supra). Cell pellets of culture were extracted at various days with 4M guanidine hydrochloride as described. Aliquots were then exhaustively dialyzed against 50 mM Tris-HCl (pH 7.4) using a microdialysis unit (Bethesda Research Laboratory, Gaithersburg, Md.) prior to assay.

Assay for Proteoglycan

In order to show the specificity of the peptide fragments of one embodiment on degradation of type II collagen as compared with proteoglycan, primarily aggrecan, sulphated glycosaminoglycan (GAG) content was determined in both the α-chymotrypsin and proteinase K digests for total proteoglycan (predominantly aggrecan) content in cell pellets, cartilage explants and also in conditioned media. Measurement of release of proteoglycan into media was tested using dimethylene blue dye binding as previously described (Dahlberg, L. et al. (2000) supra).

Assay for DNA Content

In order to show the effect of the peptide fragments of one embodiment of the invention was related to cell content, DNA content was measured in proteinase K digests of cell pellets as previously described (Nelson, F. et al. (1998) supra).

Statistical Analysis

One-way analysis of variance (ANOVA) was used to assess the measured variables. Comparisons between two groups were performed by Student's t-test. P<0.05 was considered significant.

### EXAMPLES

### Example 1

### Effects of CNBr Peptide Fragments of type II Collagen on Collagenase Induced Cleavage of type II Collagen in Bovine Pellet Culture

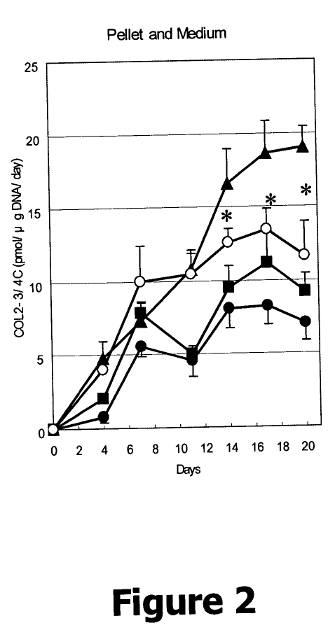

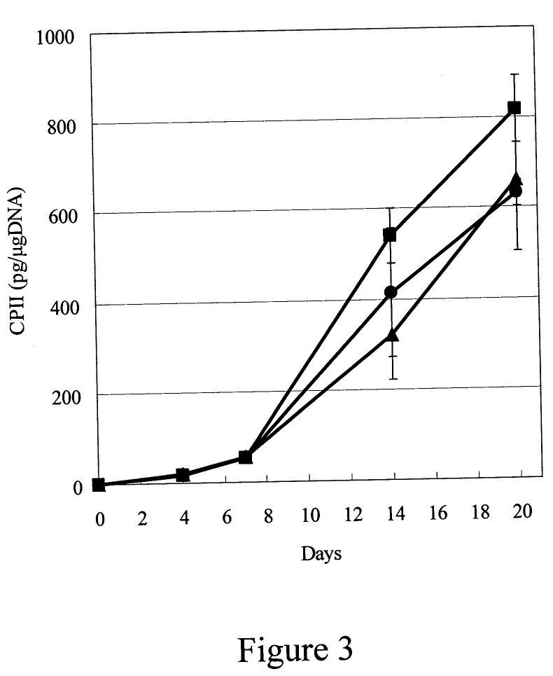

In control cultures, collagenase-cleaved type II collagen content, measured as COL2-3/4C epitope, remained constant during most of the culture period (see FIG. 2). Addition of a mixture of all the CNBr fragments, at 1 and 10 μM from day 0, caused a significant progressive increase in cleaved type II collagen content in cultures (cell pellet plus medium) in a dose-dependent manner on up to day 20 when measurements ceased. The increase in the tissue caused by CNBr fragments was maximal on day 20. All the CNBr fragments were added at 1 μM (▪) and 10 μM (▴) to serum-free media from day 0. Control cultures (●) were monitored without any additives. Collagenase-cleaved type II collagen content was determined in α-chymotrypsin extracts and conditioned media by enzyme-linked immunosorbent assay (ELISA) using a specific antibody to COL2-3/4Cₛₕₒᵣₜ epitope. One-way ANOVA confirmed a significant effect of the concentration of the CNBr fragments on resulting total COL2-3/4 content in cell pellets and media (see FIG. 2).

To confirm the involvement of collagenase, especially MMP-13, in type II collagen breakdown caused by CNBr fragments, articular chondrocyte pellets were cultured from day 0 in media containing all the CNBr fragments at 10 μM with (O) or without 10 nM RS 102,481, a preferential inhibitor of MMP-13 (FIG. 2). In view of the Ki values for MMP-1, MMP-8 and MMP-13, the synthetic inhibitor at 10 nM preferentially inhibits MMP-13 (Billinghurst et al. (2000) supra, and Dahlberg, L. et al. (2000) supra). While treatment with 10 μM CNBr fragments resulted in the highest increase in COL2-3/4C epitope generated by collagenase cleavage of type II collagen compared with control on day 10, addition of RS102,481 with CNBr fragments suppressed the elevation of the epitope present in pellets and medium (data not shown).

### Example 2

### Effects of CNBr Peptide Fragments of Type II Collagen on Denaturation and Synthesis of Type II Collagen in Pellet Culture

No significant differences in c-propeptide contents (reflective of type II collagen synthesis) were seen between cultures with and without treatment with all the CNBr fragments at 1 and 10 μM up to day 20 although CNBr fragments caused a significant increase in denaturation of type II collagen (COL2-3/4m epitope in α chymotrypsin digest) on days 17 and 20 at 10 μM (data not shown). Type II collagen content (COL2-3/4m epitope in α-chymotrypsin and protease K digests) also increased progressively up to day 11, but thereafter, CNBr peptide fragments suppressed an increase in type II collagen content with a significant reduction of the content compared with control on day 20 (data not shown).

### Example 3

### Effects of Peptide Fragments on Proteoglycan and DNA Content in Pellet Culture

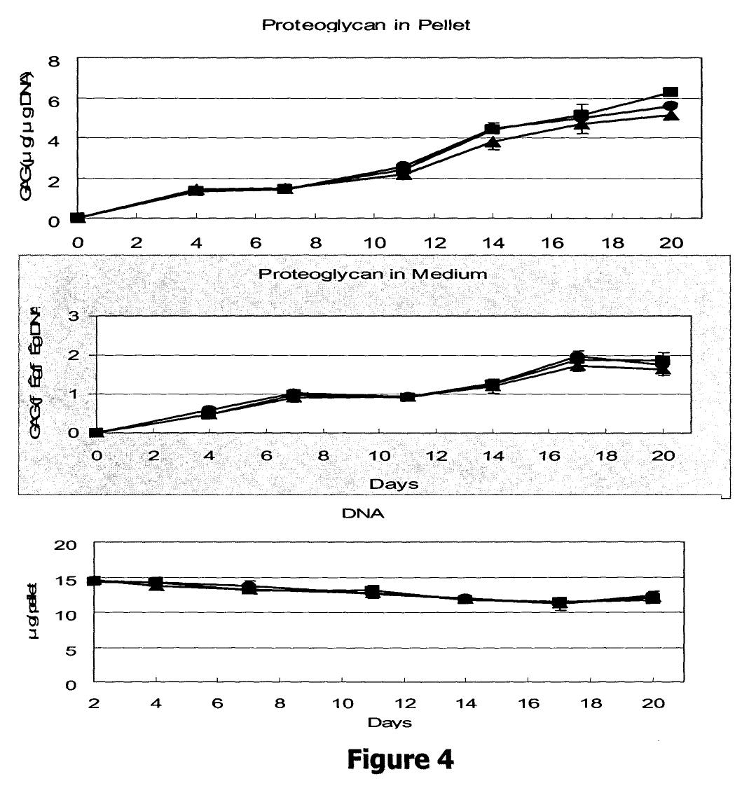

Control cultures showed progressive increases in proteoglycan content in pellets during the whole culture period (FIG. 4A). Addition of all the CNBr fragments at 1 or 10 μM had no clear effect on proteoglycan content. Proteoglycan release into media in control cultures showed a progressive elevation and reached maximal levels by day 17. CNBr fragments caused no detectable effect on proteoglycan release into media (FIG. 4B).

DNA content showed a steady decline from approximately 14 μg/pellet to 11-12 μg/pellet during the whole culture period. Addition of 1 or 10 μM CNBr fragments had no effect on DNA content (FIG. 4C).

### Example 4

### The CB12 Peptide of type II Collagen Induces Collagenase Mediated Cleavage of type II Collagen in Bovine Explant Culture.

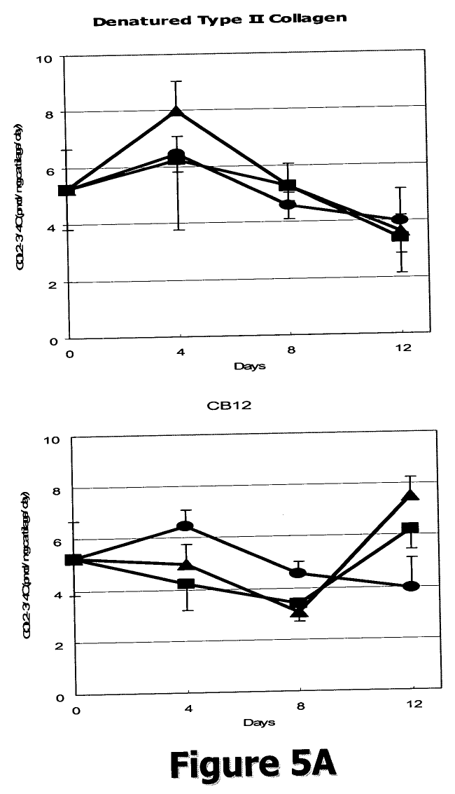

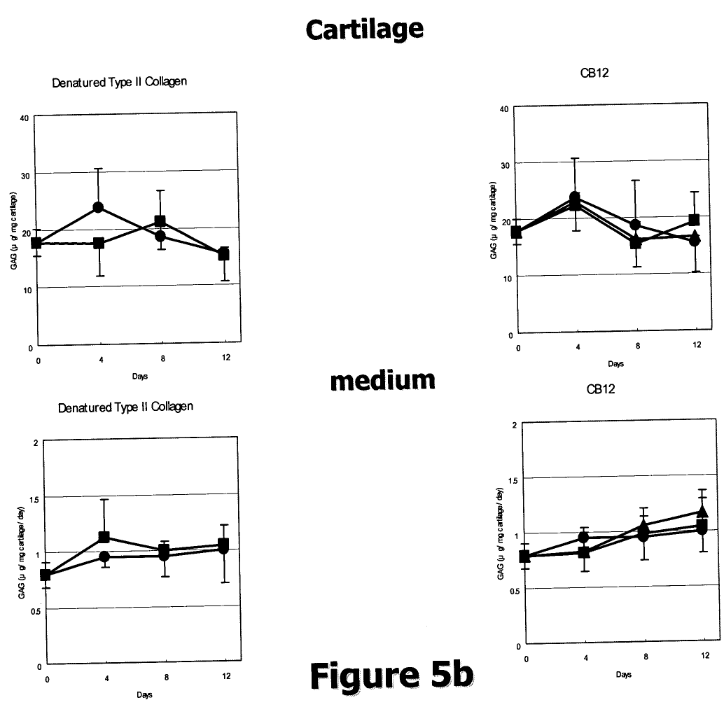

FIG. 5 shows a time course of changes in collagenase-cleaved type II collagen content in both cartilage and medium with treatment with denatured type II collagen and the CB12 peptide of type II collagen isolated by HPLC. These forms of the collagen molecule were added at 0.1 (▪) and 1 μM (▴) from day 0. Control cultures (●) were without any additives. Values are mean±SD for 4 determinates. One way ANOVA confirmed a significant effect of CB12 on the COL2-3/4C content in medium and cartilage on day 12. There was no effect of denatured collagen (FIG. 5A). In FIG. 5B, it can be seen that addition of denatured collagen and the CB12 peptide had no effect on proteoglycan (GAG) contents in pellet cultures and on GAG-release into culture media.

### Example 5

### Subpeptides of type II Collagen CB12 Peptide Induce Collagenase Mediated Cleavage of type II Collagen in Bovine Articular Cartilage.

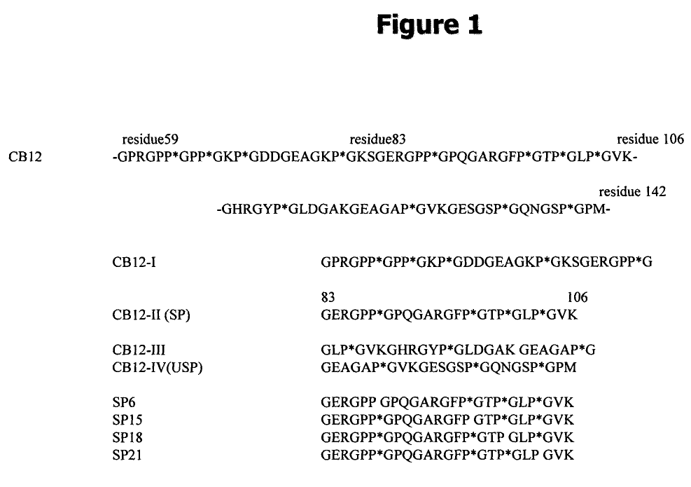

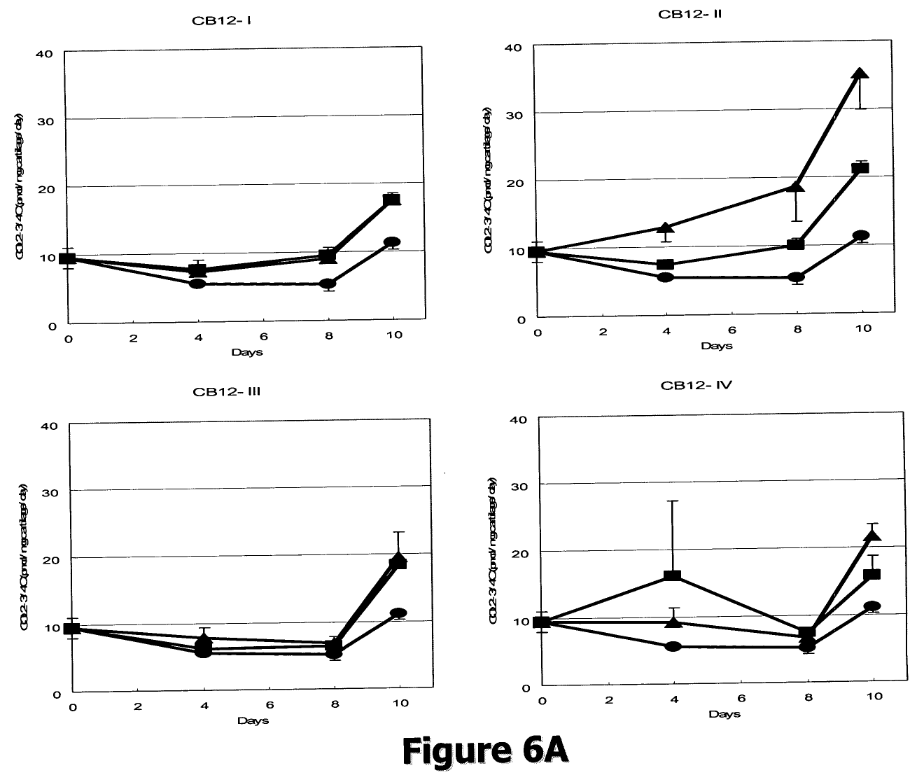

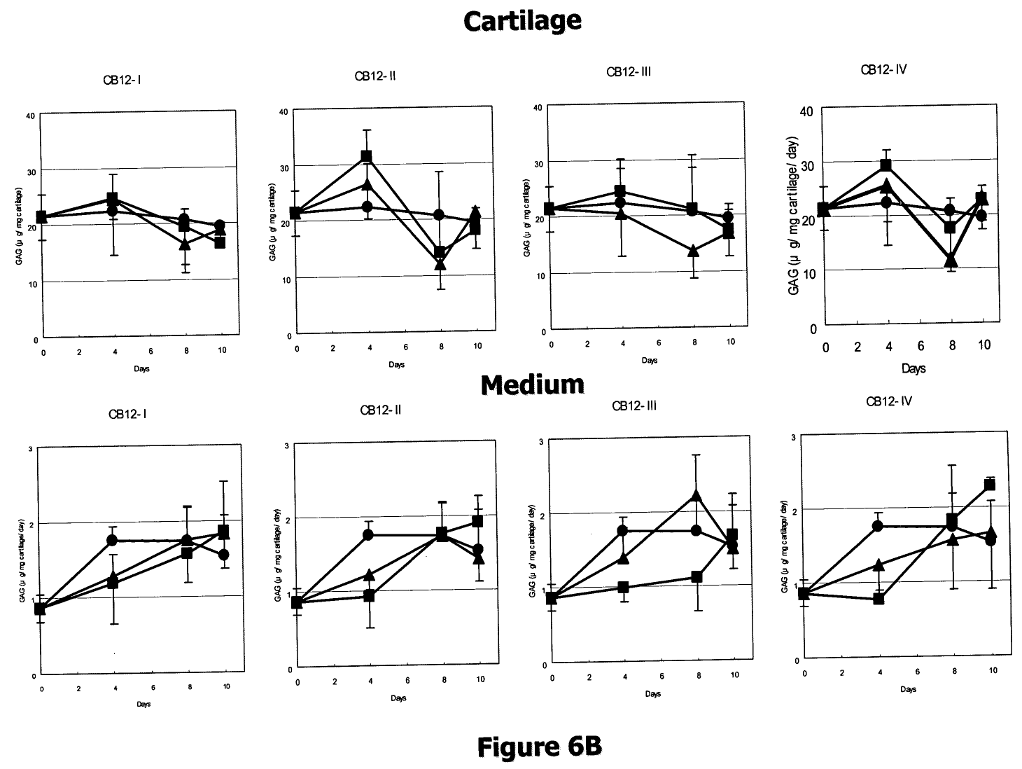

Subpeptides of the CB12 peptide (see FIG. 1) were tested to determine which of them induced type II collagen cleavage by collagenases in chondrocytes from adult bovine articular cartilage. FIG. 6A shows a representative time-dependent inductions of total collagenase cleavage of type II collagen by subpeptides CB12-I, -II, -III, and IV at 1 and 10 μM in a dose-dependent manner. Induction was most pronounced with the CB12-II peptide. CB12-I, CB12-III and CB12-IV had less activity. There was no significant effect in any of the peptides, CB12-I, CB12-II, CB12-III or CB12-IV on proteoglycan breakdown (FIG. 6B).

### Example 6

### CB12-II (SP) Peptide Fragment of Type II Collagen Induces Collagenase Mediated Cleavage of Type II Collagen in Human Explant Culture

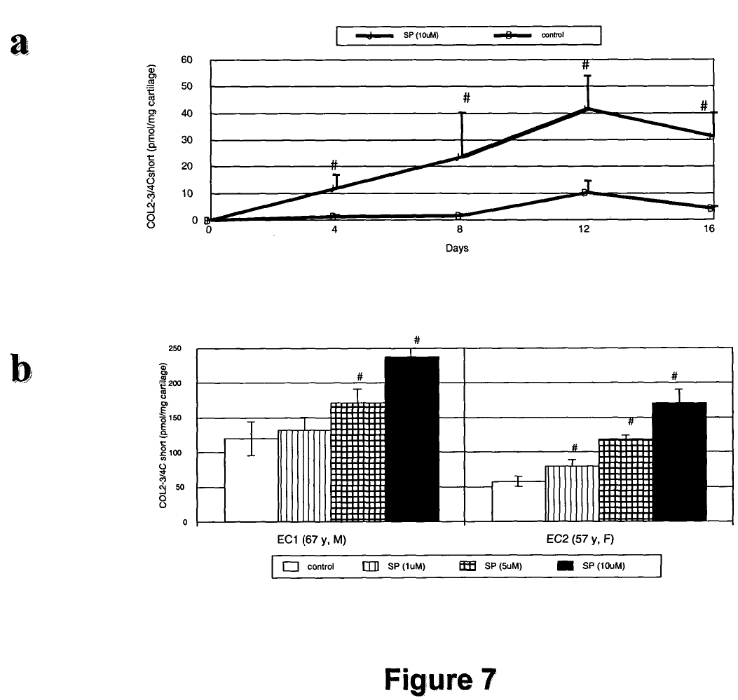

FIG. 7 shows that compared with extracellular matrix in pellet cultures, mature human articular cartilage contains higher levels of both type II collagen and proteoglycan. This makes detection of COL2-3/4C easier in articular cartilage explant culture as compared with pellet culture. A further advantage is the ability to focus on cartilage degradation in explant culture whereas pellet cultures allow chondrocytes to continue to actively synthesize cartilage matrix. Most importantly, in explant cultures the relationship of the chondrocyte to its matrix is already established and homeostasis is in place to maintain a healthy cartilage.

The CB12-II peptide (SP) at 10 μM caused a significant progressive increase in COL2-3/4 epitope production in cartilage and medium on day 12 (FIG. 7A). The induction of cleavage of type II collagen occurred in a dose dependent manner with increasing activity seen at 1 μM (in one donor), 5 μM and 10 μM in both donors (FIG. 7B).

### Example 7

### Hydroxylation of CB12-II (SP) Peptide Affects Induction of Collagenase Mediated Cleavage of Type II Collagen in Human Explant Culture

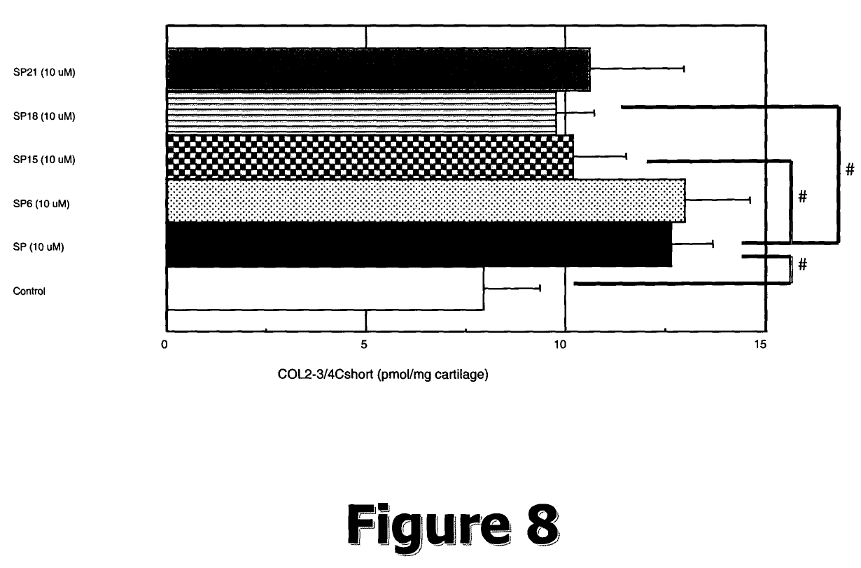

Synthetic peptides of CB12-II (SP) were synthesized with variable intrachain proline hydroxylation in the “Y” position of Gly-X-Y where “Y” is a proline (FIG. 1). Cleavage was clearly enhanced in the presence of CB12-II (SP) in the culture. Removal of hydroxylation at single proline residues 88 (SP6) or 103 (SP21) had no effect on activity by day 16 (FIG. 8). Removal of hydroxylation at residues 97 (SP15) or residue 100 (SP18) reduced potency as shown in FIG. 8. Thus hydroxylation of the peptide influences its activity.

### Example 8

### Induction of MMP Expression and Collagenase Activity by Peptide CB12-II (SP) in Human Chondrocyte Culture

Isolated human chondrocytes in culture were also incubated with peptide fragment CB12-II and the level of gene expression (mRNA by RT-PCR) compared with expression induced by incubation of IL-1 plus TNF-α. Chondrocytes were incubated with either CB12-II or TNF-α/IL-1β for 24 hours. Amplification of mRNA yielded distinct bands of the expected length for MMP-1, MMP-13, and MMP-3 (data not shown). MMPs were all upregulated by the cytokines as compared to a control of GAPDH. MMP-13 was weakly upregulated by CB12-II. MMP-1 was clearly induced by CB12-II.

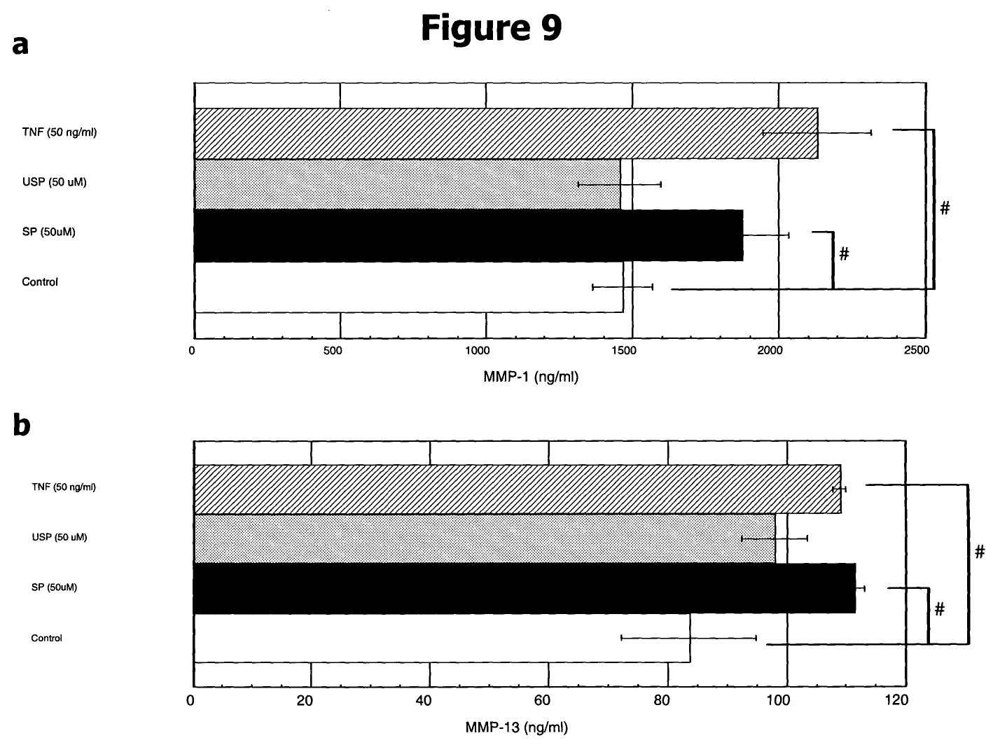

Protein levels of MMP-1 and MMP-13 were also tested by incubating isolated human chondrocytes for 48 hours in high density culture (10⁶ cells/ml) with CB12-II, negative control peptide USP, or cytokine TNF-α, as discussed above. MMP-1 and MMP-13 secretion into medium from the cells was measured using ELISA (see FIGS. 9A and 9B). CB12-II as well as TNFα induced both MMP-1 (FIG. 9A) and MMP-13 (FIG. 9B) secretion significantly higher than no peptide or and the negative control peptide (USP).

### Example 9

### Inhibition of Peptide Fragment Induction of type II Collagen Degradation in Human Explant Culture

An MMP-13 preferential inhibitor, RS102,481, was tested for inhibition of collagenase cleavage of type II collagen at 10 nM. See Billinghurst et al. (2000) supra and Dahlberg, L. et al. (2000) supra. This inhibitor was able to partially inhibit the increase in collagenase activity induced by CB12-II in normal human articular cartilage (data not shown).

### Example 10

### Effects of Synthetic Peptide CB12-I (SP) on Proteoglycan Cleavage in Human Explant Culture

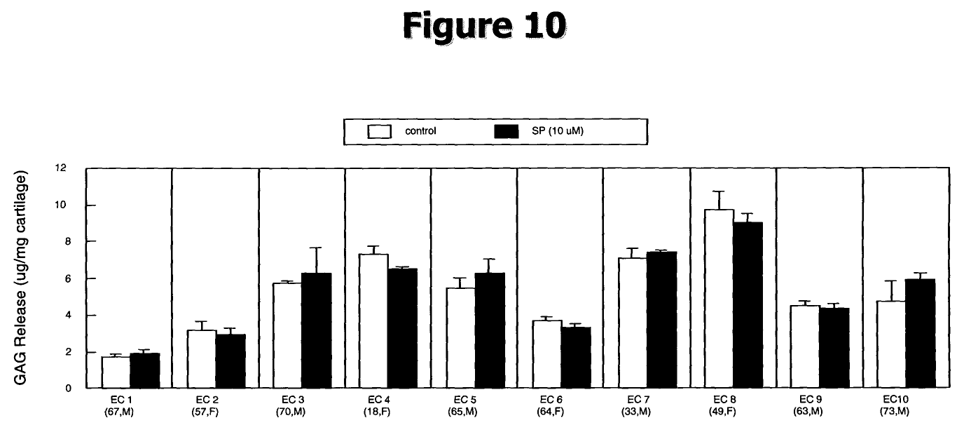

In order to examine any effects of SP on proteoglycan catabolism in normal human articular cartilage, cumulative proteoglycan (mainly aggrecan) release into medium and proteoglycan content in cartilage were determined by DMMB (1,9-dimethylmethyleneblue) dye assay. FIG. 10 demonstrates that peptide did not induce proteoglycan release in any patient nor did it decrease proteoglycan content in cartilage explants (data not shown). There was no significant difference in cumulative proteoglycan release between SP-treated and control specimens.

### Example 11

### Identification of Receptor Specific for Peptide Fragment CB12-II (SP)

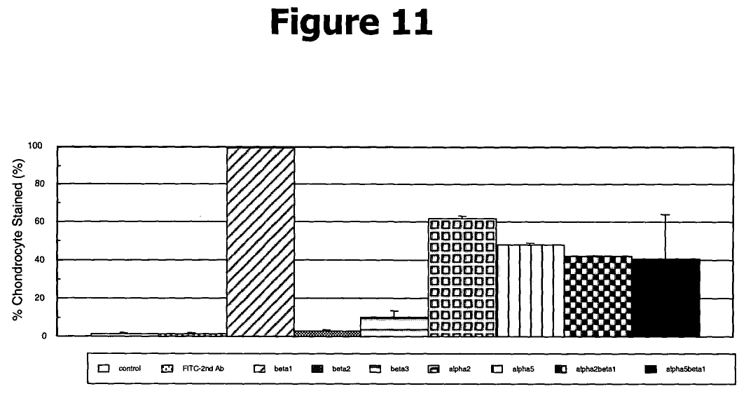

In order to seek evidence for a chondrocyte cell surface receptor mediated binding of CB12-II, anti-integrin antibodies were used to determine whether they could compete for binding of CB12-II. First, we showed by FACScan analysis that β1, α2, α5, α2β1 and α5β1 integrin subunits were expressed on a proportion of the freshly isolated human chondrocytes (see FIG. 11).

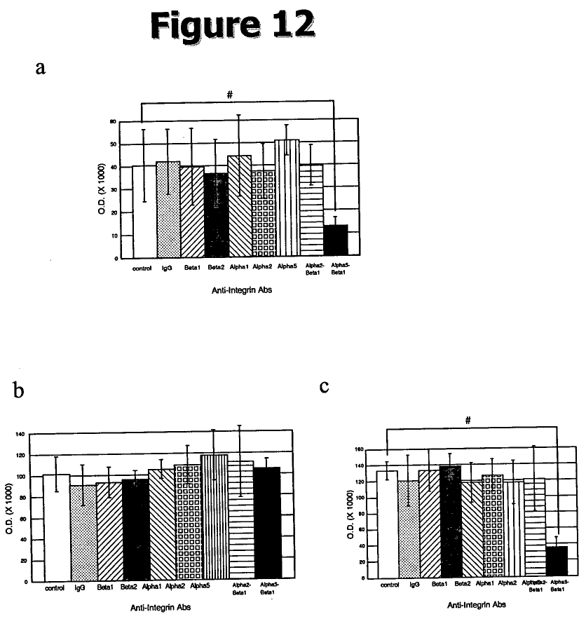

We used a well based cell-peptide attachment assay to examine peptide interaction with chondrocytes and demonstrate the involvement of integrin receptors in the autoregulation of type II collagen degradation by fragments of type II collagen. Anti-α5β1 integrin antibodies significantly inhibited adhesion of isolated human chondrocytes to the CB12-1′-coated plate (FIG. 12A). These antibodies had no significant effect on chondrocyte binding to the a plate coated with a peptide CB12-IV (USP) (see FIG. 12B). Antibodies to α1, α2, α5, and α2α1 had no consistent effect on binding. As a positive control, we also showed that anti-α5β1 antibodies inhibited adhesion of isolated human chondrocytes to human fibronectin which as been previously shown to bind to the α5β1 integrin. (FIG. 12C).

### Example 12

### CB12-II Activation of ERK1/2 MAP Kinases Pathway in Human Chondrocyte Culture

The activation of the degradative pathway initiated by CB12-II was tested by monitoring MAP kinase signaling pathway phosphorylation of ERK1/2 (p42/44) (data not shown).

First-passaged confluent human chondrocytes were treated with CB12-II (SP) (10 μM), USP (10 μM), anti-α2β1and α5β1 antibodies, anti-integrin α2β1 blocking antibody, and IL-1β or TNF-α. Cell lysates were immunoblotted using antibody that detects phosphorylated p-ERK1I/2 or p-MEK. In some experiments, U0126 at 10 μM, a specific inhibitor of an upstream molecule, MEK1/2, that activates ERK1/2, and SB203580 at 10 μM, that inhibits p38 MAPK activity, were added to the culture 1 h before peptides, antibodies, and cytokines. Herbimycin A, cytochalasin D, and wortmannin were also used as inhibitors.

ERK1/2 was phosphorylated within 5 min after adding CB12-II at 10 μM, and reached maximum at 15-30 min. At the concentration of 1, 10 and 50 μM, CB12-II induced phosphorylation of ERK 1/2 dose-dependently 15 min after the peptide was added to culture medium. In addition, CB12-II, at 10 μM, induced phosphorylation of ERK1/2 more than USP at the same concentration. Both anti-α2β1 and anti-α5β1 antibodies induced phosphorylation of ERK1/2 after 15 min incubation. Furthermore, phosphorylation of ERK1/2 induced by CB12-II at 15 min was inhibited by U0126, Cytochalasin D, or Herbimicin A, but not by SB203580.

### Example 13

### Upregulation of Various Genes in the Induction of Normal Bovine and Human Articular Cartilage Matrix Degradation and Chondrocyte Hypertrophy.

The CB-12 peptide fragment induces the expression of various genes in induced normal bovine and human articular cartilage matrix degradation which are characteristic of chondrocyte hypertrophy. These genes include genes involved in terminal differentiation such as COLX, MMP-9, TGF-B1, 1HH, MMP-13, CBFA1, SOX 9 and proliferation, for example bFGF and pTHrP and caspase-3. Other genes that are up-regulated include MT1-MMP, IL-1B, MMP-1.

Addition of the peptide SP (CB12-II) to explant cultures of both adult human and bovine articular cartilages resulted in rapid induction of the above genes within a 24-48 hr period. MMP-13 expression was maximal by 12 days. Gene expression was determined by RT-PCR analyses of mRNA in the manner used to detect expression of MMPs and cytokines described elsewhere. We also used a TUNEL staining kit (Roche) to detect apoptosis by staining 7.5 μm thick cryostat sections of articular cartilage with terminal deoxynucleotidyl transferase-mediated dUTP nick end labeling. A fluorescein apoptosis detection system was used to show apoptosis.

### Example 14

### Uses of Antibodies that Recognise Different Sequences and Epitope Contained Within the Sequence of CB12-II Peptide

Antibodies to the sequence incorporated in CNBr peptide CB12 and more specifically antibodies recognizing epitopes contained within the sequence CB12-II have been prepared to specific sequences within CB12-II. They can be used to detect the denaturation of type II collagen such as occurs in osteoarthritis. They do not react with triple helical collagen. Such antibodies have properties which are closely related to those of the mouse monoclonal antibody COL2-3/4m (Hollander, A. P. et al. (1994a) supra) However, they recognize different sequences and epitopes which detection of which by immunoassay may be of value in the preparation of immunoassay to study and detect the release of type II collagen degradation products in body fluids such as tissue extracts, serum, synovial fluid and urine. The fragments recognized by the antibodies to CB12-II may recognize peptide degradation products that are present in greater amounts in sera of patients with arthritis. Therefore they may be of value in identifying patients at risk for rapid or slow progression of disease, those responding to therapy designed to arrest cartilage degradation, and those at risk for disease who are exhibiting early preclinical changes prior to clinical presentation of arthritis (note that we have a patent filed and issued for antibodies COL2-3/4m and COL²-3/4C_{long mono}-C2C) that are used in detection of collagen fragments in sera. These new assays may be more useful for some of the above indications, just as combinations of assays e.g., COL2-3/4C_{long mono} and COL2-3/4Cₛₕₒᵣₜ) are of value in prognosis of disease progression in OA but single assays are not prognostic.

What is claimed is:

1. An isolated or purified peptide comprising an amino acid sequence selected from the group consisting of:
(a) GPRGPPGPPGKPGDDGEAGKPGKSGERGPPGPQGARGFPGTPGLPGVKGHR GYPGLDGAKGEAGAPGVKGESGSPGQNGSPGGPM (cb 12);
(b) GPRGPPGPPGKPGDDGEAGKPGKSGERGPPG (cb 12-I);
(c) ERGPPGPQGARGFPGTPGLPGVK (cb12-II);
(d) GLPGVKGHRGYPGLDGAKGEAGAPG (CB12-III);
(e) GEAGAPGVKGESGSPGQNGSPGPM (CB12-IV);
(f) GERGPPGPQGARGFP*GTP*GLP*GVK wherein the * denotes sites of hydroxylation. (pro6);
(g) GERGPP*GPQGARGFPGTP*GLP*GVK wherein the * denotes sites of hydroxylation. (pro15);
(h) GERGPP*GPQGARGFP*GTPGLP*GVK wherein the * denotes sites of hydroxylation. (pro18); and
(i) GERGPP*GPQGARGFP*GTP*GLPGVK wherein the * denotes sites of hydroxylation. (pro21)
or a fragment or conservatively substituted variant thereof, wherein said peptide is effective in altering the rate of degradation of type II collagen or the rate of chondrocyte hypertrophy.

2. The peptide fragment of claim 1, wherein said peptide is further modified by hydroxylation.

3. The peptide fragment of claim 2, wherein said peptide is hydroxylated at one or more of the proline or lysine residues of said peptide.

4. The peptide fragment of claim 3, wherein said peptide is hydroxylated at one or more proline residues located within the sequence Gly-X-Pro, where X indicates any amino acid.

5. The peptide fragment of claim 3, wherein said peptide is hydroxylated at one or more lysine residues located within the sequence Gly-X-Lys, where X indicates any amino acid.

6. A peptide fragment consisting essentially of an amino acid sequence denoted CB12:
GPRGPPGPPGKPGDDGEAGKPGKSGERGPPGPQGARGFPGTPGLPGVKGHRGY PGLDGAKGEAGAPGVKGESGSPGQNGSPGGPM.

7. The peptide fragment of claim 6, wherein said peptide is further modified by hydroxylation.

8. The peptide fragment of claim 7, wherein said peptide is hydroxylated at one or more of the proline or lysine residues of the peptide.

9. The peptide fragment of claim 8, wherein said peptide is hydroxylated at one or more proline residues located within the sequence Gly-X-Pro, where X indicates any amino acid.

10. The peptide fragment of claim 8, wherein said peptide is hydroxylated at one or more lysine residues located within the sequence Gly-X-Lys, where X indicates any amino acid.

11. The peptide fragment of claim 7, wherein said peptide consists essentially of GPRGPP* GPP*GKP*GDDGEAGKP*GKSGERGPP*GPQGARGFP*GTP*GLP*GV KGHRGYPGLDGAKGEAGAP*GVKGESGSP*GQNGSP*GGPM and wherein the * denotes sites of hydroxylation.

12. A mimetic of said peptide fragment of claim 6.

13. A mimetic of said peptide fragment of claim 11.

14. An inhibitor of said peptide fragment of claim 6.

15. An inhibitor of said peptide fragment of claim 1 1.

16. A peptide fragment consisting essentially of an amino acid sequence denoted CB12-II: GERGPPGPQGARGFPGTPGLPGVK.

17. The peptide fragment of claim 16, wherein said peptide is further modified by hydroxylation.

18. The peptide fragment of claim 17, wherein said peptide is hydroxylated at one or more of the proline or lysine residues of the peptide.

19. The peptide fragment of claim 18, wherein said peptide is hydroxylated at one or more proline residues located within the sequence Gly-X-Pro, where X indicates any amino acid.

20. The peptide fragment of claim 18, wherein said peptide is hydroxylated at one or more lysine residues located within the sequence Gly-X-Lys, where X indicates any amino acid.

21. The peptide fragment of claim 17, wherein said peptide consists essentially of GERGPP*GPQGARGFP*GTP*GLP*GVK.

22. A mimetic of said peptide fragment of claim 16.

23. A mimetic of said peptide fragment of claim 21.

24. An inhibitor of said peptide fragment of claim 16.

25. An inhibitor of said peptide fragment of claim 21.

26. The peptide fragment of claim 16, wherein said peptide consists essentially of GERGPPGPQGARGFP*GTP*GLP*GVK (Pro6) and wherein the * denotes sites of hydroxylation.

27. A mimetic of said peptide fragment of claim 26.

28. An inhibitor of said peptide fragment of claim 26.

29. The peptide fragment of claim 16, wherein said peptide consists essentially of GERGPP*GPQGARGFPGTP*GLP*GVK (Prol 5) and wherein the * denotes sites of hydroxylation.

30. A mimetic of said peptide fragment of claim 29.

31. An inhibitor of said peptide fragment of claim 29.

32. The peptide fragment of claim 16, wherein said peptide consists essentially of GERGPP*GPQGARGFP*GTPGLP*GVK (Prol 8) and wherein the * denotes sites of hydroxylation.

33. A mimetic of said peptide fragment of claim 32.

34. An inhibitor of said peptide fragment of claim 32.

35. The peptide fragment of claim 16, wherein said peptide consists essentially of GERGPP*GPQGARGFP*GTP*GLPGVK (Pro21) and wherein the * denotes sites of hydroxylation.

36. A mimetic of said peptide fragment of claim 35.

37. An inhibitor of said peptide fragment of claim 35.

38. A peptide fragment consisting essentially of an amino acid sequence denoted as CB12-I: GPRGPPGPPGKPGDDGEAGKPGKSGERGPPG.

39. The peptide fragment of claim 38, wherein said peptide is further modified by hydroxylation.

40. The peptide fragment of claim 39, wherein said peptide is hydroxylated at one or more of the proline or lysine residues of the peptide.

41. The peptide fragment of claim 40, wherein said peptide is hydroxylated at one or more proline residues located within the sequence Gly-X-Pro, where X indicates any amino acid.

42. The peptide fragment of claim 40, wherein said peptide is hydroxylated at one or more lysine residues located within the sequence Gly-X-Lys, where X indicates any amino acid.

43. The peptide fragment of claim 38, wherein said peptide consists essentially of GPRGPP*GPP*GKP*GDDGEAGKP*GKSGERGPP*G and wherein the * denotes sites of hydroxylation.

44. A mimetic of said peptide fragment of claim 38.

45. An inhibitor of said peptide fragment of claim 38.

46. A mimetic of said peptide fragment of claim 43.

47. An inhibitor of said peptide fragment of claim 43.

48. A peptide fragment wherein said peptide consists essentially of an amino acid sequence denoted as CB12-III: GLPGVKGHRGYPGLDGAKGEAGAPG.

49. The peptide fragment of claim 48, wherein said peptide is further modified by hydroxylation.

50. The peptide fragment of claim 49, wherein said peptide is hydroxylated at one or more of the proline or lysine residues of the peptide.

51. The peptide fragment of claim 50, wherein said peptide is hydroxylated at one or more proline residues located within the sequence Gly-X-Pro, where X indicates any amino acid.

52. The peptide fragment of claim 50, wherein said peptide is hydroxylated at one or more lysine residues located within the sequence Gly-X-Lys, where X indicates any amino acid.

53. The peptide fragment of claim 48, wherein said peptide consists essentially of GLP*GVKGHRGYP*GLDGAKGEAGAP*G and wherein the * denotes sites of hydroxylation.

54. A mimetic of said peptide fragment of claim 48.

55. An inhibitor of said peptide fragment of claim 48.

56. A mimetic of said peptide fragment of claim 53.

57. An inhibitor of said peptide fragment of claim 53.

58. A peptide fragment consisting essentially of an amino acid sequence denoted as CB12-IV: GEAGAPGVKGESGSPGQNGSPGPM.

59. The peptide fragment of claim 58, wherein said peptide is further modified by hydroxylation.

60. The peptide fragment of claim 59, wherein said peptide is hydroxylated at one or more of the proline or lysine residues of the peptide.

61. The peptide fragment of claim 60, wherein said peptide is hydroxylated at one or more proline residues located within the sequence Gly-X-Pro, where X indicates any amino acid.

62. The peptide fragment of claim 60, wherein said peptide is hydroxylated at one or more lysine residues located within the sequence Gly-X-Lys, where X indicates any amino acid.

63. The peptide fragment of claim 58, wherein said peptide consists essentially of GEAGAP*GVKGESGSP*GQNGSP*GPM and wherein the * denotes sites of hydroxylation.

64. A mimetic of said peptide fragment of claim 58.

65. An inhibitor of said peptide fragment of claim 58.

66. A mimetic of said peptide fragment of claim 63.

67. An inhibitor of said peptide fragment of claim 63.

68. A peptide as in any one of claims 1, 6, 16, 38, 48 or 58, wherein 1-5 acids of the peptide sequence have been replaced using conservative substitutions and wherein said peptide is effective in altering the rate of degradation of type II collagen or the rate of chondrocyte hypertrophy.

69. A peptide which has at least 80% homology to a peptide as in any one of claims 1, 6, 16, 38, 48 or 58, and wherein said peptide is effective in altering the rate of degradation of type II collagen or the rate of chondrocyte hypertrophy.

70. A peptide dimer consisting of two peptides wherein each peptide is selected from the group of peptides in claim 1.

71. The peptide dimer of claim 70, wherein said peptide dimer is a homodimer or a heterodimer.

72. A peptide trimer consisting of three peptides wherein each peptide is selected from the group of peptides of claim 1.

73. The peptide trimer of claim 72, wherein said peptide trimer is a homotrimer or a heterotrimer.

74. A pharmaceutical composition comprising a pharmaceutically effective carrier and at least one of the inhibitors of any one of claims 14, 15, 24, 25, 28, 31, 34, 37, 45, 47, 55, 57, 65, or 67.

75. Use of a pharmaceutical composition as in claim 74, wherein said composition reduces collagen matrix turnover in mammals.

76. Use of a pharmaceutical composition according to claim 74, wherein said composition reduces collagen matrix turnover in humans.

77. A method of regulating collagen turnover comprising:
administering to a subject a pharmaceutically effective amount of said pharmaceutical composition according to claim 74.

78. Use of a pharmaceutically effective dose of said pharmaceutical composition of according to claim 74, wherein the administration of said composition reduces degradation of one or more collagen proteins.

79. A method of identifying a peptide mimetic of a peptide fragment of collagen capable of decreasing the degradation of the collagen in a biological sample comprising:
(a) screening peptide fragments of collagen, and variants thereof for the ability of the peptide fragments to bind preferentially to a specific receptor of the naturally produced peptide fragments but has a lesser ability to activate the matrix degradation pathway.

80. The method of claim 79, wherein said specific receptors are anti-integrin receptors.

81. The method of claim 79, wherein said activation of the matrix degradation pathway induces the expression of genes selected from the group consisting of COLX, MMP-9, TGF-B1, 1HH, MMP-13, CBFA1, SOX 9, bFGF, pTHrP, caspase-3, MT1-MMP, IL-1B, and MMP-1.

82. The method of claim 79, wherein said biological sample is a biological fluid selected from the group consisting of tissue extracts, synovial fluid, serum and urine.

83. An isolated or purified antibody that specifically binds to an epitope of said peptide or an antigenic fragment thereof as in claims 1, 6, 16, 38, 48 or 58.

84. The antibody of claim 83, wherein said antibody is a monoclonal or a polyclonal antibody.

85. The antibody of claim 83, wherein said antibody is used to inhibit the activity of said peptide.

86. The antibody of claim 83, wherein said antibody is used to identify inhibitors of the generation of said peptide.

87. The antibody of claim 83, wherein said antibody is used to identify a subject at risk for rapid or slow progression of a disease, responding to therapy designed to arrest cartilage degradation or at risk for a disease by showing of early pre-clinical changes prior to clinical presentation of said disease.

88. The antibody of claim 87, wherein said disease is selected from the group consisting of osteoarthritis, rheumatoid arthritis, post-traumatic osteoarthritis, idiopathic osteoarthritis, and eye disease.

89. A method of diagnosing a disease selected from the group consisting of osteoarthritis, rheumatoid arthritis, post-traumatic osteoarthritis, idiopathic osteoarthritis, and eye disease comprising contacting a sample with an antibody of claim 87.

90. The antibody of claim 83, wherein said antibody is used to detect the release of type II collagen degradation products in body fluids selected from the group consisting of tissue extracts, serum, synovial fluid, and urine.

91. A method of inhibiting chondrocyte hypertrophy in a subject comprising administering to said subject a pharmaceutically effective amount of said antibody of claim 83, whereby said hypertrophy is inhibited.

92. A method of screening for a compound capable of inhibiting collagen breakdown comprising:
(a) incubating said test compound in vitro with an extract containing collagen;
(b) adding a compound known to increase degradation of collagen; and
(c) selecting said compound capable of decreasing the degradation of collagen as compared with said known compound alone.
